SEQUENCE LISTING (3 sequences)

SEQ ID NO: 1 - DNA, 21 bp, Artificial Sequence
  siRNA inhibitor of PERK gene
caaacuguauaacgguuuatt

SEQ ID NO: 2 - DNA, 1968 bp, Homo sapiens
atggccgaggcgcctcaggtggtggagatcgacccggacttcgagccgctgccccggccgcgctcgtgcacctggccgctgcccaggccggagtttagccagtccaactcggccacctccagcccggcgccgtcgggcagcgcggctgccaaccccgacgccgcggcgggcctgccctcggcctcggctgccgctgtcagcgccgacttcatgagcaacctgagcttgctggaggagagcgaggacttcccgcaggcgcccggctccgtggcggcggcggtggcggcggcggccgccgcggccgccaccggggggctgtgcggggacttccagggcccggaggcgggctgcctgcacccagcgccaccgcagcccccgccgcccgggccgctgtcgcagcacccgccggtgccccccgccgccgctgggccgctcgcggggcagccgcgcaagagcagctcgtcccgccgcaacgcgtggggcaacctgtcctacgccgacctcatcaccaaggccatcgagagctcggcggagaagcggctcacgctgtcgcagatctacgagtggatggtcaagagcgtgccctacttcaaggataagggtgacagcaacagctcggcgggctggaagaattcaattcgtcataatctgtccctacacagcaagttcattcgtgtgcagaatgaaggaactggaaaaagttcttggtggatgctcaatccagagggtggcaagagcgggaaatctcctaggagaagagctgcatccatggacaacaacagtaaatttgctaagagccgaagccgagctgccaagaagaaagcatctctccagtctggccaggagggtgctggggacagccctggatcacagttttccaaatggcctgcaagccctggctctcacagcaatgatgactttgataactggagtacatttcgccctcgaactagctcaaatgctagtactattagtgggagactctcacccattatgaccgaacaggatgatcttggagaaggggatgtgcattctatggtgtacccgccatctgccgcaaagatggcctctactttacccagtctgtctgagataagcaatcccgaaaacatggaaaatcttttggataatctcaaccttctctcatcaccaacatcattaactgtttcgacccagtcctcacctggcaccatgatgcagcagacgccgtgctactcgtttgcgccaccaaacaccagtttgaattcacccagcccaaactaccaaaaatatacatatggccaatccagcatgagccctttgccccagatgcctatacaaacacttcaggacaataagtcgagttatggaggtatgagtcagtataactgtgcgcctggactcttgaaggagttgctgacttctgactctcctccccataatgacattatgacaccagttgatcctggggtagcccagcccaacagccgggttctgggccagaacgtcatgatgggccctaattcggtcatgtcaacctatggcagccaggcatctcataacaaaatgatgaatcccagctcccatacccaccctggacatgctcagcagacatctgcagttaacgggcgtcccctgccccacacggtaagcaccatgccccacacctcgggtatgaaccgcctgacccaagtgaagacacctgtacaagtgcctctgccccaccccatgcagatgagtgccctggggggctactcctccgtgagcagctgcaatggctatggcagaatgggccttctccaccaggagaagctcccaagtgacttggatggcatgttcattgagcgcttagactgtgacatggaatccatcattcggaatgacctcatggatggagatacattggattttaactttgacaatgtgttgcccaaccaaagcttcccacacagtgtcaagacaacgacacatagctgggtgtcaggctga

SEQ ID NO: 3 - DNA, 2022 bp, Homo sapiens
atggcagaggcaccggcttccccggccccgctctctccgctcgaagtggagctggacccggagttcgagccccagagccgtccgcgatcctgtacgtggcccctgcaaaggccggagctccaagcgagccctgccaagccctcgggggagacggccgccgactccatgatccccgaggaggaggacgatgaagacgacgaggacggcgggggacgggccggctcggccatggcgatcggcggcggcggcgggagcggcacgctgggctccgggctgctccttgaggactcggcccgggtgctggcacccggagggcaagaccccgggtctgggccagccaccgcggcgggcgggctgagcgggggtacacaggcgctgctgcagcctcagcaaccgctgccaccgccgcagccgggggcggctgggggctccgggcagccgaggaaatgttcgtcgcggcggaacgcctggggaaacctgtcctacgcggacctgatcacccgcgccatcgagagctccccggacaaacggctcactctgtcccagatctacgagtggatggtgcgttgcgtgccctacttcaaggataagggcgacagcaacagctctgccggctggaagaactccatccggcacaacctgtcactgcatagtcgattcatgcgggtccagaatgagggaactggcaagagctcttggtggatcatcaaccctgatggggggaagagcggaaaagccccccggcggcgggctgtctccatggacaatagcaacaagtataccaagagccgtggccgcgcagccaagaagaaggcagccctgcagacagcccccgaatcagctgacgacagtccctcccagctctccaagtggcctggcagccccacgtcacgcagcagtgatgagctggatgcgtggacggacttccgttcacgcaccaattctaacgccagcacagtcagtggccgcctgtcgcccatcatggcaagcacagagttggatgaagtccaggacgatgatgcgcctctctcgcccatgctctacagcagctcagccagcctgtcaccttcagtaagcaagccgtgcacggtggaactgccacggctgactgatatggcaggcaccatgaatctgaatgatgggctgactgaaaacctcatggacgacctgctggataacatcacgctcccgccatcccagccatcgcccactgggggactcatgcagcggagctctagcttcccgtataccaccaagggctcgggcctgggctccccaaccagctcctttaacagcacggtgttcggaccttcatctctgaactccctacgccagtctcccatgcagaccatccaagagaacaagccagctaccttctcttccatgtcacactatggtaaccagacactccaggacctgctcacttcggactcacttagccacagcgatgtcatgatgacacagtcggaccccttgatgtctcaggccagcaccgctgtgtctgcccagaattcccgccggaacgtgatgcttcgcaatgatccgatgatgtcctttgctgcccagcctaaccagggaagtttggtcaatcagaacttgctccaccaccagcaccaaacccagggcgctcttggtggcagccgtgccttgtcgaattctgtcagcaacatgggcttgagtgagtccagcagccttgggtcagccaaacaccagcagcagtctcctgtcagccagtctatgcaaaccctctcggactctctctcaggctcctccttgtactcaactagtgcaaacctgcccgtcatgggccatgagaagttccccagcgacttggacctggacatgttcaatgggagcttggaatgtgacatggagtccattatccgtagtgaactcatggatgctgatgggttggattttaactttgattccctcatctccacacagaatgttgttggtttgaacgtggggaacttcactggtgctaagcaggcctcatctcagagctgggtgccaggctga

(19) United States
(12) Patent Application Publication
(10) Pub. No.: US 2014/0227289 A1
(43) Pub. Date: Aug. 14, 2014

(54) COMPOUNDS AND METHODS FOR TREATING INSULIN RESISTANCE SYNDROME

(71) Applicants: Stephen Michael Cohen, Singapore (SG); Wei Zhang, Singapore (SG)

(72) Inventors: Stephen Michael Cohen, Singapore (SG); Wei Zhang, Singapore (SG)

(21) Appl. No.: 14/117,620

(22) PCT Filed: May 14, 2012
(86) PCT No.: PCT/SG2012/000169

§ 371 Date: Feb. 14, 2014

(30) Foreign Application Priority Data
May 13, 2011 (SG) 2011034782

(51) Int. Cl. C07K 16/40; C07D 401/14; C12N 15/113

(52) U.S. Cl. CPC C07K 16/40; C12N 15/1137; C07D 401/14
USPC 424/158.1; 514/44 A; 544/324; 536/24.5; 530/389.1; 514/275

(57) ABSTRACT

The present invention relates to a method of treating or preventing insulin resistance syndrome in an animal body by administering an inhibitor of protein kinase RNA-like endoplasmic reticulum kinase (PERK) gene, or a functional variant thereof, or an inhibitor of PERK protein or a functional variant thereof or a method of reducing activity of transcription factors of the FOXO family (Foxo 1, 3a, 4 and 6) by administering an inhibitor of protein kinase RNA-like endoplasmic reticulum kinase (PERK) gene, or a functional variant thereof, or an inhibitor of PERK protein or a functional variant thereof. The present invention also relates to different compounds and methods for using PERK gene or PERK protein.

### CROSS-REFERENCE TO RELATED APPLICATIONS

This application claims the benefit of priority of Singapore patent application no. 201103478-2, filed May 13, 2011, the contents of it being hereby incorporated by reference in its entirety for all purposes.

### FIELD OF THE INVENTION

The present invention relates to biochemistry and medical applications of biochemical molecules.

### BACKGROUND OF THE INVENTION

Insulin resistance is when the cells of the body become resistant to the effects of insulin. Accordingly, the body provides a smaller than expected biological response to a given dose of insulin and thus, a higher amount of insulin is required in order to be effective. Insulin resistance may lead to certain diseases. For example, Type 2 diabetes is associated with resistance to insulin leading to elevated blood glucose and elevated insulin levels. Typically, insulin resistance precedes the development of Type 2 diabetes. Obesity-related insulin resistance underlies metabolic syndrome, which is characterized by excess abdominal fat, and can also lead to the development of diabetes. Insulin resistance is thus an important medical problem.

The insulin signal transduction pathway has been extensively studied. In brief, binding of insulin to the extracellular portion of the transmembrane insulin receptor triggers a cascade of molecular events within the cell, leading to activation of PI3 Kinase (PI3K). Activated PI3K increases the level of the phosphatidylinositol (3,4,5)-triphosphate (PIP(3,4,5)P₃) protein at the intracellular portion of the cell membrane, in turn leading to activation of the protein kinase known as AKT (or protein kinase B (PKB)). Activation of AKT triggers a chain of sequences, leading to the stimulation of glucose uptake into the cell and the promotion of glucose storage in the cell as glycogen.

However in an insulin resistant subject, AKT activity will be lowered. Hence, increasing AKT activity could be used to counter insulin resistance by stimulating glucose uptake. One way to increase AKT activity would be to inhibit the activity of the lipid phosphatase and tensin homolog (PTEN), which opposes PI3K activity. However, PTEN has been identified as a tumor suppressor gene, so it is likely that a drug reducing PTEN activity might pose an increased cancer risk. Furthermore, hyperactivation of AKT is also widely found in cancer. Accordingly, manipulation of the insulin-PI3K-AKT pathway would pose excessive risks to be used as a therapy for a chronic disease.

There is therefore a need to provide an alternative therapy for insulin resistance that overcomes, or at least ameliorates, one or more of the disadvantages described above. There is a need to provide further drugs that can be used to treat insulin resistance or related diseases.

### SUMMARY OF THE INVENTION

According to a first aspect, there is provided a method of treating or preventing insulin resistance syndrome in an animal body by administering an inhibitor of protein kinase RNA-like endoplasmic reticulum kinase (PERK) gene, or a functional variant thereof, or an inhibitor of PERK protein or a functional variant thereof.

According to a second aspect, there is provided a method of reducing activity of transcription factors of the FOXO family (Foxo1, 3a, 4 and 6) by administering an inhibitor of protein kinase RNA-like endoplasmic reticulum kinase (PERK) gene, or a functional variant thereof, or an inhibitor of PERK protein or a functional variant thereof.

According to a third aspect, there is provided siRNA directed against the nucleic acid transcribed from the PERK gene.

According to a fourth aspect, there is provided an antibody, or a functional variant thereof, or a fragment of the antibody capable of binding to PERK protein.

According to a fifth aspect, there is provided a pharmaceutical composition comprising an inhibitor of PERK gene or a functional variant thereof, or an inhibitor of PERK protein or a functional variant thereof.

According to a sixth aspect, there is provided a method of identifying a compound that modulates expression of PERK gene in a cell, the method comprising:
a. exposing cells expressing the PERK gene with a test compound;
b. determining the expression level of the PERK gene in the cells which were exposed to the test compound under (a);
c. comparing the level of expression of the PERK gene determined under (b) with the expression of the PERK gene in control cells which were not exposed to the test compound; wherein a difference in the expression level between the cells under (b) compared to the control cells identifies the compound that modulates expression of the PERK gene in a cell.

According to a seventh aspect, there is provided a method of identifying a compound that modulates the amount or activity of PERK protein comprised in a cell, the method comprising:
a. exposing cells expressing PERK protein with a test compound;
b. determining the amount or activity of PERK protein in the cells which were exposed to the test compound under (a);
c. comparing the amount or activity of PERK protein determined under (b) with the activity of PERK protein in control cells not exposed to the test compound; wherein a difference in the amount or activity of PERK protein between the cells under (b) compared to the control cells identifies the compound that modulates the amount of PERK protein in the cells.

According to an eighth aspect, there is provided a method of identifying a compound that modulates the amount of PERK protein comprised in a cell, the method comprising:
a. exposing cells expressing PERK protein with a test compound;
b. determining the amount of PERK protein in the cells which were exposed to the test compound under (a);
c. comparing the amount of PERK protein determined under (b) with the amount of PERK in control cells not exposed to the test compound; wherein a difference in the amount of PERK protein between the cells under (b) compared to the control cells identifies the compound that modulates the amount of PERK protein in the cells.

According to a ninth aspect, there is provided a prognostic method for determining the receptiveness of a patient suffering from insulin resistance syndrome for a treatment with a PERK inhibitor, wherein the method comprises identifying and determining the PERK activity in the patient, wherein an increased PERK activity indicates that the person may be receptive for a treatment with a PERK inhibitor.

According to a tenth aspect, there is provided a prognostic method for determining the receptiveness of a patient suffering from insulin resistance syndrome for a treatment with a PERK inhibitor, wherein the method comprises measuring activity of a protein kinase AKT and/or PI3Kinase activity in a subject, wherein in comparison to a control a lowered AKT activity and/or lowered PI3Kinase activity indicates that the patient may be receptive for a treatment with a PERK inhibitor.

According to an eleventh aspect, there is provided a prognostic method for determining the receptiveness of a patient suffering from insulin resistance syndrome for a treatment with a PERK inhibitor, wherein the method comprises measuring the relative levels of phosphorylation on the AKT site and on the PERK site(s), wherein a lower ratio of AKT site phosphorylation to PERK site phosphorylation indicates that the patient is receptive for a treatment with a PERK inhibitor.

According to a twelfth aspect, there is provided a kit for use in treating or preventing insulin resistance syndrome in a patient, said kit comprises one of the following selected from the group consisting of a siRNA as disclosed herein, an antibody as disclosed herein, an organic molecule as disclosed herein, and a pharmaceutical composition as disclosed herein.

According to a thirteenth aspect, there is provided a kit for determining whether a patient suffering from insulin resistance syndrome is receptive for a treatment with a PERK inhibitor, wherein the kit comprises:
a. antibodies specific to one or more of the AKT phosphorylation site(s) on one or more of the human FOXO proteins; and
b. antibodies specific for one or more of the PERK phosphorylation site(s) on one or more of the human FOXO proteins or for one or more of the PERK phosphorylation site(s) on PERK protein or on eIF2α.

According to a fourteenth aspect, there is provided the use of an inhibitor of protein kinase RNA-like endoplasmic reticulum kinase (PERK) gene, or a functional variant thereof, or an inhibitor of the activity of PERK protein, or a functional variant thereof, in the manufacture of a medicament for the treatment or prevention of insulin resistance syndrome.

According to a fifteenth aspect, there is provided the use of an inhibitor of protein kinase RNA-like endoplasmic reticulum kinase (PERK) gene, or a functional variant thereof, or an inhibitor of the activity of PERK protein or a functional variant thereof, in the manufacture of a medicament to reduce activity of transcription factors of the FOXO family (Foxo1, 3a, 4 and 6) for the treatment or prevention of insulin resistance syndrome.

### BRIEF DESCRIPTION OF THE DRAWINGS

The invention will be better understood with reference to the detailed description when considered in conjunction with the non-limiting examples and the accompanying drawings, in which:

FIG. 1(a) shows a schematic diagram of the connection between insulin signaling and FOXO in a cell. The binding of insulin to the transmembrane insulin receptor triggers a chain of events which increases AKT activity. AKT phosphorylates the FOXO transcription factors in the cell nucleus and brings the phosphorylated FOXO out of the nucleus into the cytoplasm, thereby preventing the expression of the target genes of FOXO, such as Bim, GADD45 and CHOP. FIG. 1(b) shows a schematic diagram of the connection between FOXO and PERK in a healthy cell, an insulin resistant cell and a cell treated for insulin resistance. AKT is less active in an insulin resistant cell, thereby reducing AKT-mediated phosphorylation of FOXO. This results in an increase in nuclear FOXO localization and up-regulation of FOXO targets by PERK-mediated phosphorylation of FOXO which promotes nuclear localization. Consequently, a cell can be treated to reduce PERK activity, thereby down-regulating the expression of FOXO targets and offsetting the effects of insulin resistance.

FIGS. 2(a) to 2(d) show the nuclear and/or cytoplasmic localization of FOXO-GFP in samples (a) to (d) referred to in Example 1. Sample (a) refers to the control cells, sample (b) refers to insulin treated cells, sample (c) refers to endoplasmic reticulum (ER) stress induced cells and sample (d) refers to ER stress induced cells that have PERK depleted by RNA interference (RNAi). From FIG. 2(a), it can be seen that the FOXO-GFP protein was predominantly nuclear, but insulin treatment shifts the location of FOXO towards the cytoplasm as seen in FIG. 2(b). From FIG. 2(c), it can be seen that ER stress counteracts the effects of insulin by increasing the proportion of nuclear FOXO, but depletion of PERK by RNAi increases cytoplasmic FOXO as seen in FIG. 2(d).

FIG. 3A shows the levels of expression of the FOXO target, 4E-BP (a regulator of overall translation levels in cells), and PERK in Drosophila wing disc tissue expressing the nub-Gal4 transgene alone (“Nub”) or together with an upstream activation sequence (UAS)-PERK transgene (“Nub>PERK”) referred to in Example 2A. The data is presented as fold change relative to the level in the “Nub” control sample. It can be seen that overexpression of PERK mRNA increased the levels of 4E-BP in “Nub>PERK” as compared to “Nub”.

FIG. 3B shows the relative eye size in Drosophila expressing the GMR-Gal4 transgene (“ctrl”) referred to in Example 2B. The total eye areas were measured in pixels from digital microscopic images using standardized magnification and the pixels were plotted in arbitrary units for each sample. PERK was then depleted by RNA interference (RNAi) and this sample is denoted as “PERK RNAi”. Overexpression of an upstream activation sequence (UAS)—FOXO transgene was done to the control fly (“FOXO”). PERK was further depleted by RNAi from the “FOXO” fly and this sample is denoted as “FOXO,PERK RNAi”. As seen in FIG. 3B, depletion of PERK had no effect on the control fly, when comparing “ctrl” with “PERK RNAi”. However, when comparing “FOXO” with “FOXO,PERK RNAi”, it can be seen that depletion of PERK counteracted the effects of FOXO overexpression, as evidenced by an increase in relative eye size.

FIG. 4A shows normalized luciferase levels of the FOXO luciferase reporter referred to in Example 3A in human MCF-7 cells. Control cells expressed a human Foxo3a luciferase reporter alone (“4FRE”). Cells were further co-transfected to express Foxo3a (“+Foxo3a”), PERK (“+PERK”), or both Foxo3a and PERK together (“+Foxo3a+PERK”). As seen in FIG. 4A, a higher level of luciferase in “+Foxo3a” indicates that expression of Foxo3a increased reporter gene expression when compared with “4FRE”. PERK expression alone had little effect on the control, when comparing “4FRE” with “PERK”. However, co-expression of PERK potentiated the effects of Foxo3a in the sample “+Foxo3a+PERK”.

FIG. 4B shows normalized luciferase levels of the FOXO luciferase reporter referred to in Example 3B in human MCF-7 cells. Control cells expressed a human Foxo1 luciferase reporter alone (“IRS”). Cells were further co-transfected to express Foxo1 (“+Foxo1”), PERK (“+PERK”), or both Foxo1 and PERK together (“+Foxo1+PERK”). As seen in FIG. 4B, a higher level of luciferase in “+PERK” as compared to “IRS” presumably indicates that overexpression of PERK was acting on endogenous Foxo1. A higher level of luciferase in “+Foxo1” indicates that expression of Foxo1 increased reporter gene expression when compared with “IRS”. Co-expression of PERK potentiated the effects of Foxo1 in the sample “+Foxo1+PERK”.

FIG. 5A shows normalized levels of mRNA transcripts of FOXO targets referred to in Example 4A: CCAAT/enhancer binding protein epsilon (CHOP), Bim (anti- or pro-apoptotic regulators), Growth Arrest and DNA Damage gene (GADD45) and PERK, in human AGS cells. Bip2, which is not a FOXO target, was also measured as a control. Non-treated human AGS cells (“ctrl”) were treated with antibiotic tunicamycin to induce ER stress (“TM”). In FIG. 5A, it can be seen that “TM” cells had significantly higher levels of Bip2, CHOP, Bim and GADD45 as compared to “ctrl” cells. Thus, it can be concluded that ER stress up-regulates FOXO activity. FIG. 5B shows normalized levels of mRNA transcripts of the FOXO targets referred to in Example 4B in human AGS cells. “TM” cells were further treated with siRNA treatment to deplete PERK (“PERK RNAi+TM”). In FIG. 5B, it can be seen from “PERK RNAi+TM” that PERK depletion down-regulates FOXO activity, thereby reducing the degree of induction of the FOXO targets. The levels of Bip2 transcripts were not affected since Bip2 is not a FOXO target.

FIG. 6 shows the nuclear and/or cytoplasmic localization of PERK in insulin treated Drosophila cells (“Control+insulin”) and ER stress induced cells (“ER stress”) referred to in Example 6. The Serine residues of Drosophila FOXO phosphorylation sites 1 and 4 were mutated to Alanine and denoted as “S66A” and “S243A” and treated with tunicamycin to induce ER stress. In FIG. 6, it can be seen that ER stress counteracted the effects of insulin when comparing “Control+insulin” and “ER stress” as nuclear localization was increased in “ER stress”. However, ER stress did not up-regulate the activity of mutated FOXO evidenced by higher cytoplasmic localization of PERK in “S66A” and “S243A”. It can thus be concluded that Drosophila FOXO phosphorylation sites 1 and 4, i.e. S66 and S243, are required to induce movement of Drosophila FOXO into the nucleus.

FIG. 7 shows normalized luciferase levels of the human Foxo1 luciferase reporter referred to in Example 7. The S303 residue of human Foxo1 phosphorylation site was mutated to Alanine (corresponding to phosphorylation site 4, S243, in Drosophila FOXO), denoted as “S303A”. Control cells were co-transfected to express the luciferase reporter and Foxo1 (“IRS+Foxo1”) and PERK (“+PERK”). The mutated “S303A” was also co-transfected to express the luciferase reporter and Foxo1 (“IRS+Foxo1 S303A”) and PERK (“+PERK”). As seen in FIG. 7, “IRS+Foxo1S303A” was slightly less effective than “IRS+Foxo1”. When Foxo1 (“IRS+Foxo1”) is co-expressed with PERK (“+PERK”), the activity of human Foxo1 was enhanced by about 2 fold. However, the effect of PERK was attenuated when co-expressed with Foxo1 S303A, evidenced by the lower levels of normalized luciferase in the “+PERK” samples. This indicates that S303 in human Foxo1 plays an important role in mediating the effects of PERK on Foxo1 activity.

FIG. 8 shows the DNA sequences of Foxo1 and Foxo3 and the corresponding phosphorylated sites and phospho-peptides in ER-stress induced human H1299 cells referred to in Example 8.

FIG. 9 shows normalized luciferase levels of the human Foxo1 luciferase reporter referred to in Example 9. The S298 residue of human Foxo1 was mutated to Alanine (corresponding to phosphorylation site 4, S243, in Drosophila FOXO), denoted as “S298A”, and tested for responsiveness to PERK. H1299 cells were transfected to express natural Foxo1 (“FOXO1”) or the S298A mutant version of Foxo1 (“S298A”) and each were co-transfected to overexpress PERK (“FOXO1+PERK” and “S298A+PERK”). As seen in FIG. 9, the activity of “S298A” was comparable to that of “FOXO1” in the reporter assay without added PERK. However, “S298A” shows a lower response to PERK overexpression (S298A+PERK”) as compared to “FOXO1+PERK”. It can thus be concluded that 5298 is one of the sites on human Foxo1 that contributes to mediating the effects of PERK on Foxo1 activity.

FIG. 10 shows normalized luciferase levels of the human Foxo1 luciferase reporter referred to in Example 10. Human H1299 cells were transfected to express human Foxo1 (“FOXO1”), co-transfected to express PERK (“FOXO1+PERK”) and both were treated with PERK kinase inhibitor (“FOXO1+inhibitor” and “FOXO1+PERK+inhibitor”). In FIG. 10, it can be seen from “FOXO1” and “FOXO1+PERK” that PERK potentiates the effect of Foxo1 alone. However, partial inhibition of PERK activity reduced the level of Foxo1 activity when comparing “FOXO1+PERK” and “FOXO1+PERK+inhibitor”. It can thus be concluded that PERK kinase inhibitor partially inhibits PERK activity, thereby down-regulating Foxo1 activity.

FIG. 11A shows the normalized expression levels of endogenous Foxo1 mRNA targets in MCF-7 cells that were serum starved to remove insulin (“control with serum”) and treated with a PI3K inhibitor to further reduce AKT activity (“serum starved+LY”) to simulate extreme insulin resistance, as described in Example 11A. The cells were further treated with PERK kinase inhibitor (“serum starved+LY+PERK inhibition”). The endogenous FOXO targets are BIM, Cyclin G2, IRS-2, p27KIP1 and PDK4. In FIG. 11A, it can be seen that reduction of AKT activity increases Foxo1 activity, evidenced by the substantial increase of target gene expression of “serum starved+LY” as compared to “control with serum”. Further, PERK inhibition counteracted the effects of AKT reduction, thereby reducing the target gene expression of “serum starved+LY+PERK inhibition” as compared to “serum starved+LY”. FIG. 11B shows the normalized expression levels of FOXO targets in HEPG2 cells instead, referred to in Example 11B. FIG. 11B confirms that PERK inhibition counteracted the effects of AKT reduction and reduced target genes expression. The FOXO targets are p27, PDK, Cyclin G, PCK2, p21 and INSR. However, PERK inhibition did not reduce the expression of rp132 and mActin, which are not FOXO targets.

FIG. 12A shows the normalized expression levels of Foxo1 target genes in HEPG2 liver cells treated with palmitate to induce insulin resistance. The data was normalized to Kinesin mRNA levels and to the level of the rp132 control mRNAs. The Foxo1 target genes measured were PDK4 and PCK1. In FIG. 12A, it can be seen that the palmitate treated cells had substantially higher levels of PDK4 and PCK1 expression. However, the levels of rp123 and mActin, which are not Foxo1 targets, were not substantially altered. FIG. 12B shows the normalized expression levels of Foxo1 targets, PDK4 and PCK1, in palmitate treated cells and palmitate treated cells further treated with PERK kinase inhibitor. The data was normalized to Kinesin mRNA levels and to the level of the rp132 control mRNAs. In FIG. 12B, it can be seen that PERK inhibition decreased the expression of PDK4 and PCK1. However, the levels of rp123 and mActin, which are not Foxo1 targets, were not substantially altered. It can thus be concluded that a reduction of Foxo1 activity is a consequence of PERK inhibition in cells induced with insulin resistance.

### DETAILED DESCRIPTION OF THE PRESENT INVENTION

It is known that an increase in the activity of the protein kinase known as AKT (or protein kinase B (PKB)) could be used to treat insulin resistance. However, hyperactivation of AKT is also widely found in cancer.

The present inventors have now surprisingly found alternative methods and compounds for treating insulin resistance and related diseases. Specifically, the present inventors have surprisingly found a way to downregulate the activity of the transcription factors of the Forkhead box class 0 (FOXO) family to provide a means to treat insulin resistance and related diseases.

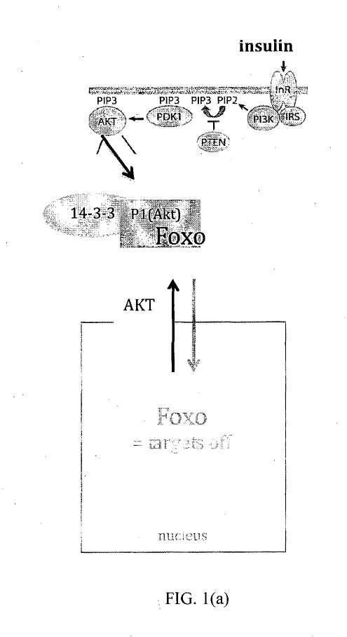

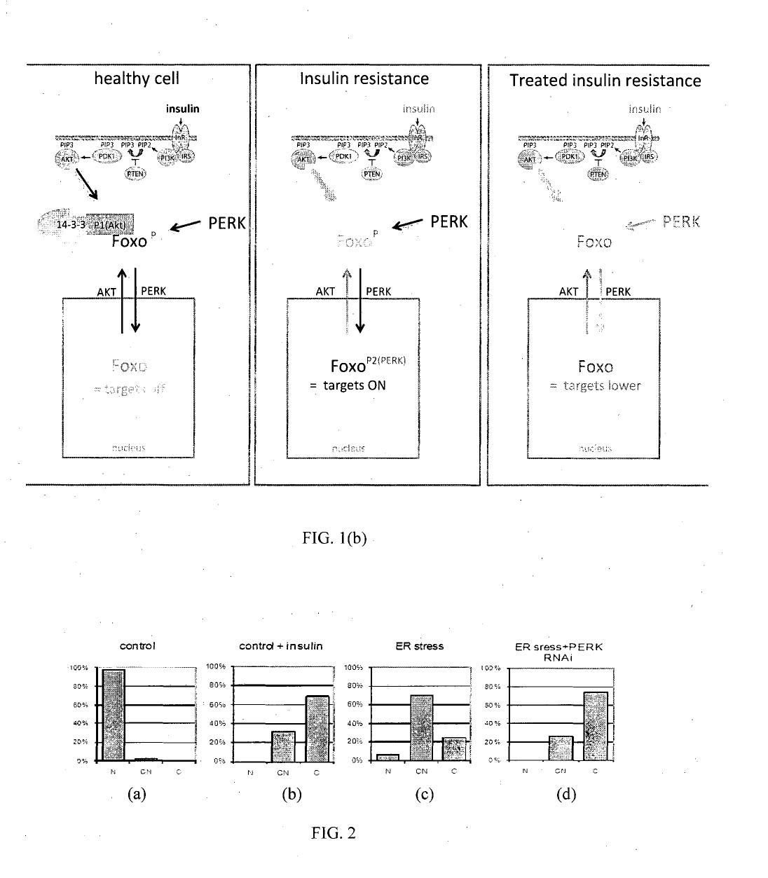

One of the targets of AKT is the FOXO transcription factors (Foxo1, 3a, 4 and 6) whose nuclear localization is regulated by AKT-mediated phosphorylation. The connection between insulin signaling and FOXO in a cell is schematically shown in FIG. 1(a). Referring to FIG. 1(a), the binding of insulin to the transmembrane insulin receptor (InR) triggers a cascade of molecular events leading to the activation of PI3 Kinase (PI3K). Activated PI3K increases the level of the phosphatidylinositol (3,4,5)-triphosphate (PIP(3,4,5)P₃) protein at the cell membrane, in turn leading to activation of AKT. Phosphorylation of Foxo1 by AKT is known to create a binding site for the 14-3-3 proteins, which brings FOXO out of the nucleus and into the cytoplasm, thereby leading to the retention of the phosphorylated FOXO in the cytoplasm. The effects of AKT are comparable on all FOXO family members.

For the avoidance of doubt, it is to be noted that the use of the term “FOXO” herein refers to all members of the FOXO transcription factor family, while references to a specific family member will be defined by number e.g. Foxo1.

From FIG. 1(a), it is clear that an outcome of the insulin signal is the increase in AKT activity, which increases AKT-mediated FOXO phosphorylation and increases retention of FOXO in the cytoplasm, where it cannot act as a transcription factor to induce the expression of its target genes in the nucleus. Thus, insulin signaling can be described as a reduction of FOXO activity and the consequential reduction of the expression of genes whose expression is regulated by FOXO. Insulin resistance can therefore be described as increased nuclear FOXO activity, leading to increased expression of FOXO regulated (targeted) genes.

However, FOXO are transcription factors and as such are not conventional druggable targets. The present inventors have now identified that protein kinase RNA-like endoplasmic reticulum kinase (PERK), also known as eukaryotic translation initiation factor 2, α-subunit, kinase-3 (eIF2α K₃), regulates FOXO. Specifically, as seen in FIG. 1(b) it was found by the inventors that PERK phosphorylates FOXO, which promotes the nuclear localization of FOXO. PERK activity therefore acts in opposition to AKT because AKT increases retention of FOXO in the cytoplasm, while PERK increases retention of FOXO in the nucleus. As AKT activity stimulates glucose uptake and thus counters insulin resistance, PERK activity can therefore be seen to be representative of insulin resistance. In an insulin resistant cell, AKT is less active, thereby reducing AKT-mediated phosphorylation of FOXO. This results in an increase in nuclear FOXO localization and up-regulation of FOXO targets by PERK-mediated phosphorylation of FOXO which promotes nuclear localization. Accordingly, inhibition of PERK provides a means to treat or prevent insulin resistance and related diseases by reducing nuclear FOXO activity.

As a result, in one embodiment, there is provided a method of treating or preventing insulin resistance syndrome in an animal body by administering an inhibitor of protein kinase RNA-like endoplasmic reticulum kinase (PERK) gene, or a functional variant thereof, or an inhibitor of PERK protein or a functional variant thereof.

In another embodiment, there is provided a method of reducing activity of transcription factors of the FOXO family by administering an inhibitor of PERK gene, or a functional variant thereof, or an inhibitor of PERK protein or a functional variant thereof.

Members of the FOXO family in mammals include Foxo1, Foxo2, Foxo3, Foxo4 and Foxo6, while Foxo5 is the fish ortholog of Foxo3. Foxo3 is known as Foxo3a, while Foxo3b is a pseudogene. In one embodiment, the FOXO family members are Foxo1, 3a, 4 and 6.

As used in the context of the specification, the phrases “inhibiting the PERK gene” or “inhibitor of the PERK gene”, or variants thereof, mean that the expression of the PERK gene is decreased or absent. Further, in the context of the specification, the phrases “inhibiting the PERK protein” or “inhibitor of the PERK protein”, or variants thereof, mean that the activity of the PERK protein is decreased or absent. Absent means that there is completely no expression of the PERK gene or activity of the PERK protein. Functional variants of an inhibitor of the PERK gene within the context of the specification refers to genes which possess a biological activity (either functional or structural) that is substantially similar to the PERK gene disclosed herein. Functional variants of an inhibitor of PERK protein may be construed similarly to refer to a protein that is altered by one or more amino acids. The term “functional variant” also includes a fragment, a variant based on the degenerative nucleic acid code or a chemical derivative. A functional variant may have conservative changes, wherein a substituted amino acid or nucleic acid has similar structural or chemical properties to the replaced amino acid/nucleic acid, e.g. replacement of leucine with isoleucine. A functional variant may also have non-conservative changes, e.g. replacement of a glycine with a tryptophan, or a deletion and/or insertion of one or more amino acids, or a deletion and/or insertion of one or more nucleic acids. It is understood that the functional variant at least partially retains its biological activity, e.g. function, of the PERK gene or PERK protein, or even exhibits improved biological activity.

It is understood that the inhibition of the PERK gene decreases the expression of the PERK protein. Accordingly, as PERK phosphorylates FOXO and promotes the nuclear localization of FOXO, the inhibition of the PERK gene or inhibition of the activity of the PERK protein down-regulates the downstream phosphorylation of FOXO. Consequently, nuclear FOXO activity is down-regulated.

Insulin resistance syndrome makes up a broad clinical spectrum and is defined as any abnormalities associated with insulin resistance. Abnormalities such as the resistance to insulin, diabetes, hypertension, dyslipidemia and cardiovascular disease constitute the insulin resistance syndrome.

The insulin resistance syndrome may be diet-induced insulin resistance and/or obesity-induced insulin resistance. Diet-induced insulin resistance means that the resistance to insulin is induced by a diet high in saturated fat and carbohydrates. Obesity-induced insulin resistance means that the resistance to insulin is induced by a genetic predisposition to obesity or obesity which is due to dietary habits.

The disclosed methods may be used for treating any one of the following conditions which are caused by insulin resistance syndrome: insulin resistance, hypertension, dyslipidemia, Type 2 diabetes or coronary artery disease.

Hypertension refers to the sustained elevation of resting systolic blood pressure, diastolic blood pressure or both. Dyslipidemia refers to elevated plasma cholesterol and/or triglyceride concentration or a low high-density-lipoprotein level that contributes to the development of artherosclerosis. Coronary artery disease involves the impairment of blood flow through the coronary arteries, most commonly by atheromas.

Insulin resistance refers to a decreased ability to respond to the action of insulin, which is compensated by a downstream increase in the endogenous secretion of insulin. Over time, a person suffering from insulin resistance can develop high plasma sugar levels as the increased secretion of insulin can no longer compensate for elevated plasma sugar levels.

Insulin resistance typically precedes the development of Type 2 diabetes. In certain individuals who subsequently develop Type 2 diabetes, insulin resistance may be present for many years before the actual onset of diabetes. In Type 2 diabetes, insulin secretion is inadequate. Accordingly, Type 2 diabetes relates to relative insulin deficiency. On the other hand, Type 1 diabetes is an absolute insulin deficiency due to the destruction of insulin-producing cells in the pancreas. Accordingly, treatment of Type 1 diabetes may be linked to the restoration of insulin production. In contrast, treatment of Type 2 diabetes is linked to the restoration of sensitivity to insulin.

In general, the term “inhibition” as used in the context of the specification means a decrease in the expression of PERK gene or the activity of PERK protein and a corresponding downstream decrease in FOXO activity.

The inhibitor of any of the genes referred to herein may comprise at least one oligonucleotide or at least one antibody or at least one inorganic molecule or at least one organic molecule.

The oligonucleotide may be an interfering ribonucleic acid (iRNA), or protein nucleic acid (PNA) or locked nucleic acid (LNA).

The term “oligonucleotide” generally refers to a single-stranded nucleotide polymer made of more than 2 nucleotide subunits covalently joined together. Preferably between 10 and 100 nucleotide units are present, most preferably between 12 and 50 nucleotides units are joined together. The sugar groups of the nucleotide subunits may be ribose, deoxyribose or modified derivatives thereof such as 2′-O-methyl ribose. The nucleotide subunits of an oligonucleotide may be joined by phosphodiester linkages, phosphorothioate linkages, methyl phosphonate linkages or by other rare or non-naturally-occurring linkages that do not prevent hybridization of the oligonucleotide. Furthermore, an oligonucleotide may have uncommon nucleotides or normucleotide moieties. An oligonucleotide, as defined herein, is a nucleic acid, such as deoxyribonucleic acid (DNA) or ribonucleic acid (RNA), or have a combination of ribo- and deoxyribonucleotides covalently linked

The term “oligonucleotide” may also refer, in the context of the specification, to a nucleic acid analogue of those known in the art, for example Locked Nucleic Acid (LNA), or a mixture thereof. The term “oligonucleotide” includes oligonucleotides composed of naturally occurring nucleobases, sugars and internucleoside (backbone) linkages as well as oligonucleotides having non-naturally-occurring portions which function similarly or with specific improved functions. A fully or partly modified or substituted oligonucleotide is often preferred over native forms because of several desirable properties of such oligonucleotides such as for instance, the ability to penetrate a cell membrane, good resistance to extra- and intracellular nucleases, high affinity and specificity for the nucleic acid target. Methods of modifying oligonucleotides in this manner are known in the art.

In some oligonucleotides, sometimes called oligonucleotide mimetics, both the sugar and the internucleoside linkage, i.e., the backbone, of the nucleotide units are replaced with novel groups. The base units are maintained for hybridization with an appropriate nucleic acid target compound. One such oligomeric compound, an oligonucleotide mimetic that has been shown to have excellent hybridization properties, is referred to as a protein nucleic acid (PNA). In PNA compounds, the sugar-backbone of an oligonucleotide is replaced with an amide containing backbone, in particular an aminoethylglycine backbone. The nucleobases are retained and are bound directly or indirectly to aza nitrogen atoms of the amide portion of the backbone.

A further modification includes Locked Nucleic Acids (LNAs) in which the 2′-hydroxyl group is linked to the 3′ or 4′ carbon atom of the sugar ring thereby forming a bicyclic sugar moiety. The linkage is preferably a methelyne (—CH₂—)ₙ group bridging the 2′ oxygen atom and the 4′ carbon atom, wherein n is 1 or 2. The term “LNA” generally refers to a nucleotide containing one bicyclic nucleoside analogue, also referred to as a LNA monomer, or an oligonucleotide containing one or more bicyclic nucleoside analogues.

Examples of modified oligonucleotides include, but are not limited to oligonucleotides with phosphorothioate backbones and oligonucleosides with heteroatom backbones, and in particular-CH₂—NH—O—CH₂—, —CH₂—N(CH₃)—O—CH₂— [known as a methylene (methylimino) or MMI backbone], —CH₂—O—N(CH₃)—CH₂—, —CH₂—N(CH₃)—N(CH₃)—CH₂— and —O—N(CH₃)—CH₂—CH₂— [wherein the native phosphodiester backbone is represented as —O—P—O—CH₂—]. Also usable are oligonucleotides having morpholino backbone structures.

The interfering ribonucleic acid may be a small interfering ribonucleic acid (siRNA) or small hairpin ribonucleic acid (shRNA) or micro ribonucleic acid (miRNA).

Modified oligonucleotides used as interfering ribonucleic acids may also contain one or more substituted sugar moieties. Preferred oligonucleotides comprise one of the following at the 2′ position: OH; F; O-, S-, or N-alkyl; O-, S-, or N-alkenyl; O-, S- or N-alkynyl; or O-alkyl-O-alkyl, wherein the alkyl, alkenyl and alkynyl may be substituted or unsubstituted C₁ to C₁₀ alkyl or C₂ to C₁₀ alkenyl and alkynyl. Particular examples include, but are not limited to O[(CH₂)ₙO]ₘCH₃, O(CH₂)ₙOCH₃, O(CH₂)ₙNH₂, O(CH₂)ₙCH₃, O(CH₂)ₙONH₂, and O(CH₂)ₙON[(CH₂)ₙCH₃)]₂, where n and m are from 1 to about 10. Other exemplary oligonucleotides comprise one of the following at the 2′ position: C₁ to C₁₀ lower alkyl, substituted lower alkyl, alkenyl, alkynyl, alkaryl, aralkyl, O-alkaryl or O-aralkyl, SH, SCH₃, OCN, Cl, Br, CN, CF₃, OCF₃, SOCH₃, SO₂CH₃, ONO₂, NO₂, N₃, NH₂, heterocycloalkyl, heterocycloalkaryl, aminoalkylamino, polyalkylamino, substituted silyl, an RNA cleaving group, a reporter group, an intercalator, a group for improving the pharmacokinetic properties of an oligonucleotide, or a group for improving the pharmacodynamic properties of an oligonucleotide, and other substituents having similar properties. One exemplary modification includes 2′-methoxyethoxy(2¹-O—CH₂CH₂OCH₃, also known as 2′-O-(2-methoxyethyl) or 2′-MOE), i.e., an alkoxyalkoxy group.

As used herein, the term “siRNA” refers to a ribonucleic acid (RNA) or RNA analog comprising between about 10 to 50 nucleotides (or nucleotide analogs) capable of directing or mediating the RNA interference pathway. These molecules can vary in length and can contain varying degrees of complementarity to their target messenger RNA (mRNA) in the antisense strand. The term “siRNA” includes duplexes of two separate strands, i.e. double stranded RNA, as well as single strands that can form hairpin structures comprising of a duplex region. The siRNA may have a length of between about 10 to 50 nucleotides, or between about 15 to 50 nucleotides, or between about 20 to 50 nucleotides, or between about 25 to 50 nucleotides, or between about 30 to 50 nucleotides, or between about 35 to 50 nucleotides, or between about 40 to 50 nucleotides, or between about 10 to 45 nucleotides, or between about 10 to 40 nucleotides, or between about 10 to 35 nucleotides, or between about 10 to 30 nucleotides, or between about 10 to 25 nucleotides, or between about 10 to 20 nucleotides, or between about 15 to 50 nucleotides, or between about 15 to 35 nucleotides, or between about 15 to 30 nucleotides, or between about 15 to 25 nucleotides. In one embodiment, the siRNA has a length of between 15 to 30 nucleotides.

The application of siRNA to down-regulate the activity of its target mRNA is known in the art. In some embodiments, mRNA degradation occurs when the anti-sense strand, or guide strand, of the siRNA directs the RNA-induced silencing complex (RISC) that contains the RNA endonuclease Ago2 to cleave its target mRNA bearing a complementary sequence. Accordingly, the siRNA may be complementary to any portion of varying lengths on the PERK gene. The siRNA may also be complementary to the sense strand and/or the anti-sense strand of the PERK gene. Accordingly, siRNA treatment may be used to silence the PERK gene, thereby depleting the PERK protein downstream.

Hence, in one embodiment, there is provided siRNA directed against the nucleic acid transcribed from the PERK gene.

The siRNA may be directed against fragments of the nucleic acid transcribed from the PERK gene. Accordingly, the siRNA may comprise a sequence that is complementary to any fragment of the PERK gene. In one embodiment, the siRNA comprises the following sequence: 5′-CAAACUGUAUAACGGUUUATT-3′ (SEQ ID NO: 1), or functional variants thereof. Such functional variants thereof may comprise at least one modified or substituted nucleotide. Functional modifications and/or substitutions of the siRNA may be performed by methods known in the art.

The term “shRNA”, as used herein, refers to a unimolecular RNA that is capable of performing RNAi and that has a passenger strand, a loop and a guide strand. The passenger and guide strand may be substantially complementary to each other. The term “shRNA” may also include nucleic acids that contain moieties other than ribonucleotide moieties, including, but not limited to, modified nucleotides, modified internucleotide linkages, non-nucleotides, deoxynucleotides, and analogs of the nucleotides mentioned thereof.

miRNAs down-regulate their target mRNAs. The term “miRNA” generally refers to a single stranded molecule, but in specific embodiments, may also encompass a region or an additional strand that is partially (between 10% and 50% complementary across length of strand), substantially (greater than 50% but less than 100% complementary across length of strand) or fully complementary to another region of the same single-stranded molecule or to another nucleic acid. Thus, nucleic acids may encompass a molecule that comprises one or more complementary or self-complementary strand(s) or “complements” of a particular sequence comprising a molecule. For example, precursor miRNA may have a self-complementary region, which is up to 100% complementary. miRNA probes or nucleic acids of the invention can include, can be or can be at least 60%, 65%, 70%, 75%, 80%, 85%, 90%, 95%, 96%, 97%, 98%, 99% or 100% complementary to their target.

The inhibitor may comprise of at least one antibody. The term “antibody” refers to an antigen-binding protein having a basic four-polypeptide chain structure consisting of two heavy and two light chains, said chains being stabilized, for example, by interchain disulfide bonds, which has the ability to specifically bind antigen. The term “antibody” includes monoclonal antibodies (including full-length monoclonal antibodies), polyclonal antibodies, multispecific antibodies (for example, bispecific antibodies), chimeric antibodies, CDR-grafted antibodies, humanized antibodies, human antibodies and single chain antibodies (scFvs). The term “antibody” also includes anticalins, which are artificial proteins that are able to bind to antigens. Anticalins are not structurally related to antibodies and thus are a type of antibody mimetic.

PERK is a transmembrane receptor protein. Hence, fragments of PERK may be from the luminal domain or the cytosolic domain. Antibodies may be used to inhibit PERK protein such that substrate binding to PERK, or an isolated PERK fragment thereof, may partially be inhibited. The degree of inhibition of the substrate binding may differ. Accordingly, in one embodiment, there is provided an antibody, or functional variant thereof, or a fragment of the antibody capable of binding to PERK protein. In this embodiment, the antibody, or the functional variant thereof, or the fragment of the antibody may be a monoclonal or polyclonal antibody. Antibodies may be generated by methods known in the art using a PERK protein or a fragment thereof.

The term “isolated”, when used in the context of an isolated protein, or fragment of protein, refers to a protein that has been removed from its natural environment, for example, as part of an organ, tissue or organism. The term does not imply that the protein is the only protein present, but does indicate that the protein is sufficiently separated from other proteins with which it would naturally be associated with, so that it is the predominant protein present (at least 10-20% more than any other proteins). “Isolated” is not meant to exclude artificial or synthetic mixtures with other compounds or materials, or the presence of impurities that do not interfere with the fundamental activity of the protein and that may be present, for example, due to incomplete purification, addition of stabilizers, or compounding into, for example, immunogenic preparations or pharmaceutical acceptable preparations.

The term “fragment”, when used in the context of a protein, refers to a part or portion of a protein comprising fewer polypeptide chains than an intact or complete protein. Fragments can be obtained via chemical or enzymatic treatment of an intact or complete protein.

The inhibitor may comprise of at least one organic molecule or at least one inorganic molecule. The term “organic molecule” refers, for example, to any molecule that is made up predominantly of carbon and hydrogen, such as, alkanes and arylamines. The term “inorganic molecule” refers, for example, to molecules that are not organic molecules.

In one embodiment, the organic molecule is selected from the group consisting of:
5-bromo-N₄-2-pyridinyl-N₂-[3-(1,2,3,6-tetrahydro-4-pyridinyl)-1H-indol-5-yl]-2,4-pyrimidinediamine;
1-[5-(4-Amino-7-methyl-7H-pyrrolo[2,3-d]pyrimidin-5-yl)-2,3-dihydro-1H-indol-1-yl]-2-[3-fluoro-5-(trifluoromethyl)phenyl]ethanone;
1-methyl-3-[1-(phenylacetyl)-2,3-dihydro-1H-indol-5-yl]-1H-pyrazolo[3,4-d]pyrimidin-4-amine;
3-{1-[(2,5-difluorophenyl)acetyl]-2,3-dihydro-1H-indol-5-yl}-1-methyl-1H-pyrazolo[3,4-d]pyrimidin-4-amine;
3-[1-(phenylacetyl)-2,3-dihydro-1H-indol-5-yl]-1H-pyrazolo[3,4-d]pyrimidin-4-amine;
7-methyl-5-[1-(phenylacetyl)-2,3-dihydro-1H-indol-5-yl]-7H-pyrrolo[2,3-d]pyrimidin-4-amine;
3-[1-(phenylacetyl)-2,3-dihydro-1H-indol-5-yl]thieno[3,2-c]pyridin-4-amine;
3-{1-[(2,5-difluorophenyl)acetyl]-2,3-dihydro-1H-indol-5-yl}thieno[3,2-c]pyridin-4-amine;
3-[1-(phenylacetyl)-2,3-dihydro-1H-indol-5-yl]-7-(3-pyridinyl)thieno[3,2-c]pyridin-4-amine;
1-methyl-4-{1-[(3-methylphenyl)acetyl]-2,3-dihydro-1H-indol-5-yl}-1H-indazol-3-amine;
3-[1-(phenylacetyl)-2,3-dihydro-1H-indol-5-yl]-7-(4-pyridinyl)thieno[3,2-c]pyridin-4-amine;
3-{1-[(2,5-difluorophenyl)acetyl]-2,3-dihydro-1H-indol-5-yl}-7-(3-pyridinyl)thieno[3,2-c]pyridin-4-amine;
3-{1-[(2,5-difluorophenyl)acetyl]-2,3-dihydro-1H-indol-5-yl}-7-(1H-pyrazol-3-yl)thieno[3,2-c]pyridin-4-amine;
4-{1-[(2,5-difluorophenyl)acetyl]-2,3-dihydro-1H-indol-5-yl}-1-methyl-1H-indazol-3-amine;
3-[1-(phenylacetyl)-2,3-dihydro-1H-indol-5-yl]-7-(1H-pyrazol-4-yl)thieno[3,2-c]pyridin-4-amine;
7-(1-methyl-1H-pyrazol-4-yl)-3-[1-(phenylacetyl)-2,3-dihydro-1H-indol-5-yl]thieno[3,2-c]pyridin-4-amine;
3-{1-[(2-fluorophenyl)acetyl]-2,3-dihydro-1H-indol-5-yl}-1-methyl-1H-pyrazolo[3,4-d]pyrimidin-4-amine;
3-{1-[(3-fluorophenyl)acetyl]-2,3-dihydro-1H-indol-5-yl}-1-methyl-1H-pyrazolo[3,4-d]pyrimidin-4-amine;
1-methyl-3-{1-[(2-methylphenyl)acetyl]-2,3-dihydro-1H-indol-5-yl}-1H-pyrazolo[3,4-d]pyrimidin-4-amine;
1-methyl-3-{1-[(3-methylphenyl)acetyl]-2,3-dihydro-1H-indol-5-yl}-1H-pyrazolo[3,4-d]pyrimidin-4-amine;
3-[1-(phenylacetyl)-2,3-dihydro-1H-indol-5-yl]-7-(1,2,3,6-tetrahydro-4-pyridinyl)thieno[3,2-c]pyridin-4-amine;
3-(1-[3-(trifluoromethyl)phenyl]acetyl-2,3-dihydro-1H-indol-5-yl)thieno[3,2-c]pyridin-4-amine;
3-{1-[(2-chlorophenyl)acetyl]-2,3-dihydro-1H-indol-5-yl}thieno[3,2-c]pyridin-4-amine;
3-{1-[(3-chlorophenyl)acetyl]-2,3-dihydro-1H-indol-5-yl}thieno[3,2-c]pyridin-4-amine;
3-(1-[3-(methyloxy)phenyl]acetyl-2,3-dihydro-1H-indol-5-yl)thieno[3,2-c]pyridin-4-amine;
3-(1-{[2-(methyloxy)phenyl]acetyl}-2,3-dihydro-1H-indol-5-yl)thieno[3,2-c]pyridin-4-amine;
3-[1-(2-naphthalenylacetyl)-2,3-dihydro-1H-indol-5-yl]thieno[3,2-c]pyridin-4-amine;
3-[1-(phenylacetyl)-2,3-dihydro-1H-indol-5-yl]-7-(4-piperidinyl)thieno[3,2-c]pyridin-4-amine;
7-{3-[(dimethylamino)methyl]phenyl}-3-[1-(phenylacetyl)-2,3-dihydro-1H-indol-5-yl]thieno[3,2-c]pyridin-4-amine;
3-{1-[(2,5-dimethylphenyl)acetyl]-2,3-dihydro-1H-indol-5-yl}-1-methyl-1H-pyrazolo[3,4-d]pyrimidin-4-amine;
3-{1-[(3-fluoro-5-methylphenyl)acetyl]-2,3-dihydro-1H-indol-5-yl}-1-methyl-1H-pyrazolo[3,4-d]pyrimidin-4-amine;
3-{1-[(3,5-dimethylphenyl)acetyl]-2,3-dihydro-1H-indol-5-yl}-1-methyl-1H-pyrazolo[3,4-d]pyrimidin-4-amine;
5-{1-[(2,5-difluorophenyl)acetyl]-2,3-dihydro-1H-indol-5-yl} thieno[2,3-d]pyrimidin-4-amine;
3-{1-[(2,3-difluorophenyl)acetyl]-2,3-dihydro-1H-indol-5-yl}-1-methyl-1H-pyrazolo[3,4-d]pyrimidin-4-amine;
7-methyl-5-{1-[(2-methylphenyl)acetyl]-2,3-dihydro-1H-indol-5-yl}-7H-pyrrolo[2,3-d]pyrimidin-4-amine;
5-{1-[(2-fluorophenyl)acetyl]-2,3-dihydro-1H-indol-5-yl}-7-methyl-7H-pyrrolo[2,3-d]pyrimidin-4-amine;
5-{1-[(3-fluorophenyl)acetyl]-2,3-dihydro-1H-indol-5-yl}-7-methyl-7H-pyrrolo[2,3-d]pyrimidin-4-amine;
3-{1-[(2,3-difluorophenyl)acetyl]-2,3-dihydro-1H-indol-5-yl}thieno[3,2-c]pyridin-4-amine;
7-methyl-5-{1-[(3-methylphenyl)acetyl]-2,3-dihydro-1H-indol-5-yl}-7H-pyrrolo[2,3-d]pyrimidin-4-amine;
3-{1-[(3-fluoro-2-methylphenyl)acetyl]-2,3-dihydro-1H-indol-5-yl}thieno[3,2-c]pyridin-4-amine;
3-{2-[5-(4-aminothieno[3,2-c]pyridin-3-yl)-2,3-dihydro-1H-indol-1-yl]-2-oxoethyl}benzonitrile;
3-{1-[(2-fluoro-5-methylphenyl)acetyl]-2,3-dihydro-1H-indol-5-yl}-1-methyl-1H-pyrazolo[3,4-d]pyrimidin-4-amine;
3-{1-[(2,3-dimethylphenyl)acetyl]-2,3-dihydro-1H-indol-5-yl}-1-methyl-1H-pyrazolo[3,4-d]pyrimidin-4-amine;
3-{1-[(3-chlorophenyl)acetyl]-2,3-dihydro-1H-indol-5-yl}-1-methyl-1H-pyrazolo[3,4-d]pyrimidin-4-amine;
1-methyl-3-(1-{[3-(trifluoromethyl)phenyl]acetyl}-2,3-dihydro-1H-indol-5-yl)-1H-pyrazolo[3,4-d]pyrimidin-4-amine;
7-methyl-5-(1-{[3-(trifluoromethyl)phenyl]acetyl}-2,3-dihydro-1H-indol-5-yl)-7H-pyrrolo[2,3-d]pyrimidin-4-amine;
5-{1-[(3-fluoro-5-methylphenyl)acetyl]-2,3-dihydro-1H-indol-5-yl}-7-methyl-7H-pyrrolo[2,3-d]pyrimidin-4-amine;
5-{1-[(3-chlorophenyl)acetyl]-2,3-dihydro-1H-indol-5-yl}-7-methyl-7H-pyrrolo[2,3-d]pyrimidin-4-amine;
5-{1-[(2-chlorophenyl)acetyl]-2,3-dihydro-1H-indol-5-yl}-7-methyl-7H-pyrrolo[2,3-d]pyrimidin-4-amine;
7-methyl-5-(1-{[2-(methyloxy)phenyl]acetyl}-2,3-dihydro-1H-indol-5-yl)-7H-pyrrolo[2,3-d]pyrimidin-4-amine;
1-methyl-3-(1-{[3-(methyloxy)phenyl]acetyl}-2,3-dihydro-1H-indol-5-yl)-1H-pyrazolo[3,4-d]pyrimidin-4-amine;
7-methyl-5-(1-{[3-(methyloxy)phenyl]acetyl}-2,3-dihydro-1H-indol-5-yl)-7H-pyrrolo[2,3-d]pyrimidin-4-amine;
3-{1-[(2-chlorophenyl)acetyl]-2,3-dihydro-1H-indol-5-yl}-1-methyl-1H-pyrazolo[3,4-d]pyrimidin-4-amine;
1-methyl-3-(1-{[2-(methyloxy)phenyl]acetyl}-2,3-dihydro-1H-indol-5-yl)-1H-pyrazolo[3,4-d]pyrimidin-4-amine;
5-{1-[(3-chloro-5-fluorophenyl)acetyl]-2,3-dihydro-1H-indol-5-yl}-7-methyl-7H-pyrrolo[2,3-d]pyrimidin-4-amine;
3-{1-[(2,5-difluorophenyl)acetyl]-2,3-dihydro-1H-indol-5-yl}furo[3,2-c]pyridin-4-amine;
1-methyl-3-{1-[(2,3,5-trifluorophenyl)acetyl]-2,3-dihydro-1H-indol-5-yl}-1H-pyrazolo[3,4-d]pyrimidin-4-amine;
5-{1-[(2,5-dimethylphenyl)acetyl]-2,3-dihydro-1H-indol-5-yl}-7-methyl-7H-pyrrolo[2,3-d]pyrimidin-4-amine;
3-{1-[(2,5-difluorophenyl)acetyl]-2,3-dihydro-1H-indol-5-yl}-7-(1H-pyrazol-4-yl)furo[3,2-c]pyridin-4-amine;
3-{1-[(3,5-dichlorophenyl)acetyl]-2,3-dihydro-1H-indol-5-yl}-1-methyl-1H-pyrazolo[3,4-d]pyrimidin-4-amine;
5-{1-[(2,5-difluorophenyl)acetyl]-2,3-dihydro-1H-indol-5-yl}-7-methyl-7H-pyrrolo[2,3-d]pyrimidin-4-amine;
3-{1-[(2,5-difluorophenyl)acetyl]-2,3-dihydro-1H-indol-5-yl}-7-(1H-pyrazol-4-yl)thieno[3,2-c]pyridin-4-amine;
3-{1-[(3,5-difluorophenyl)acetyl]-2,3-dihydro-1H-indol-5-yl}-1-methyl-1H-pyrazolo[3,4-d]pyrimidin-4-amine;
5-{1-[(3-methylphenyl)acetyl]-2,3-dihydro-1H-indol-5-yl}-7-(4-piperidinyl)-7H-pyrrolo[2,3-d]pyrimidin-4-amine;
5-{1-[(3-methylphenyl)acetyl]-2,3-dihydro-1H-indol-5-yl}-7-(1-methyl-4-piperidinyl)-7H-pyrrolo[2,3-d]pyrimidin-4-amine;
5-{1-[(3-methylphenyl)acetyl]-2,3-dihydro-1H-indol-5-yl}thieno[2,3-d]pyrimidin-4-amine;
3-{1-[(3-fluoro-5-methylphenyl)acetyl]-2,3-dihydro-1H-indol-5-yl}furo[3,2-c]pyridin-4-amine;
3-{1-[(3-chloro-5-fluorophenyl)acetyl]-2,3-dihydro-1H-indol-5-yl}furo[3,2-c]pyridin-4-amine;
3-{1-[(2-fluoro-5-methylphenyl)acetyl]-2,3-dihydro-1H-indol-5-yl}furo[3,2-c]pyridin-4-amine;
1-methyl-3-{1-[(1-methyl-1H-pyrrol-2-yl)acetyl]-2,3-dihydro-1H-indol-5-yl}-1H-pyrazolo[3,4-d]pyrimidin-4-amine;
3-{1-[(3-chlorophenyl)acetyl]-2,3-dihydro-1H-indol-5-yl}furo[3,2-c]pyridin-4-amine;
5-{1-[(2,3-difluorophenyl)acetyl]-2,3-dihydro-1H-indol-5-yl}-7-methyl-7H-pyrrolo[2,3-d]pyrimidin-4-amine;
5-{1-[(2-fluoro-3-methylphenyl)acetyl]-2,3-dihydro-1H-indol-5-yl}-7-methyl-7H-pyrrolo[2,3-d]pyrimidin-4-amine;
5-{1-[(3-fluoro-2-methylphenyl)acetyl]-2,3-dihydro-1H-indol-5-yl}-7-methyl-7H-pyrrolo[2,3-d]pyrimidin-4-amine;
5-{1-[(2-fluoro-5-methylphenyl)acetyl]-2,3-dihydro-1H-indol-5-yl}-7-methyl-7H-pyrrolo[2,3-d]pyrimidin-4-amine;
3-{1-[(2-fluoro-3-methylphenyl)acetyl]-2,3-dihydro-1H-indol-5-yl}-1-methyl-1H-pyrazolo[3,4-d]pyrimidin-4-amine;
3-{1-[(3-fluoro-2-methylphenyl)acetyl]-2,3-dihydro-1H-indol-5-yl}-1-methyl-1H-pyrazolo[3,4-d]pyrimidin-4-amine;
5-{1-[(2,5-difluorophenyl)acetyl]-2,3-dihydro-1H-indol-5-yl}-7-(1-methyl-4-piperidinyl)-7H-pyrrolo[2,3-d]pyrimidin-4-amine;
5-{1-[(3-chloro-4-fluorophenyl)acetyl]-2,3-dihydro-1H-indol-5-yl}-7-methyl-7H-pyrrolo[2,3-d]pyrimidin-4-amine;
5-{1-[(3-chloro-2-fluorophenyl)acetyl]-2,3-dihydro-1H-indol-5-yl}-7-methyl-7H-pyrrolo[2,3-d]pyrimidin-4-amine;
3-{1-[(3-chloro-4-fluorophenyl)acetyl]-2,3-dihydro-1H-indol-5-yl}-1-methyl-1H-pyrazolo[3,4-d]pyrimidin-4-amine;
3-{1-[(3-chloro-2-fluorophenyl)acetyl]-2,3-dihydro-1H-indol-5-yl}-1-methyl-1H-pyrazolo[3,4-d]pyrimidin-4-amine;
5-{1-[(2,3-dimethylphenyl)acetyl]-2,3-dihydro-1H-indol-5-yl}-7-methyl-7H-pyrrolo[2,3-d]pyrimidin-4-amine;
1-(1-methylethyl)-3-{1-[(3-methylphenyl)acetyl]-2,3-dihydro-1H-indol-5-yl}-1H-pyrazolo[3,4-d]pyrimidin-4-amine;
2-(4-amino-3-{1-[(3-methylphenyl)acetyl]-2,3-dihydro-1H-indol-5-yl}-1H-pyrazolo[3,4-d]pyrimidin-1-yl)ethanol;
5-{1-[(3,5-dimethylphenyl)acetyl]-2,3-dihydro-1H-indol-5-yl}-7-methyl-7H-pyrrolo[2,3-d]pyrimidin-4-amine;
5-{1-[(2,5-difluorophenyl)acetyl]-2,3-dihydro-1H-indol-5-yl}-7-(4-piperidinyl)-7H-pyrrolo[2,3-d]pyrimidin-4-amine;
1-ethyl-3-{1-[(3-methylphenyl)acetyl]-2,3-dihydro-1H-indol-5-yl}-1H-pyrazolo[3,4-d]pyrimidin-4-amine;
3-{1-[(2,5-difluorophenyl)acetyl]-2,3-dihydro-1H-indol-5-yl}-7-methylfuro[3,2-c]pyridin-4-amine;
3-{1-[(2,5-difluorophenyl)acetyl]-2,3-dihydro-1H-indol-5-yl}-1-(1-methylethyl)-1H-pyrazolo[3,4-d]pyrimidin-4-amine;
5-{1-[(3,5-difluorophenyl)acetyl]-2,3-dihydro-1H-indol-5-yl}-7-methyl-7H-pyrrolo[2,3-d]pyrimidin-4-amine;
7-methyl-5-{1-[(2,3,5-trifluorophenyl)acetyl]-2,3-dihydro-1H-indol-5-yl}-7H-pyrrolo[2,3-d]pyrimidin-4-amine;
5-{1-[(3,5-dichlorophenyl)acetyl]-2,3-dihydro-1H-indol-5-yl}-7-methyl-7H-pyrrolo[2,3-d]pyrimidin-4-amine;
7-(3-azetidinyl)-5-{1-[(3-methylphenyl)acetyl]-2,3-dihydro-1H-indol-5-yl}-7H-pyrrolo[2,3-d]pyrimidin-4-amine;
5-{1-[(4-fluorophenyl)acetyl]-2,3-dihydro-1H-indol-5-yl}-7-methyl-7H-pyrrolo[2,3-d]pyrimidin-4-amine;
7-methyl-5-{1-[(4-methylphenyl)acetyl]-2,3-dihydro-1H-indol-5-yl}-7H-pyrrolo[2,3-d]pyrimidin-4-amine;
5-{1-[(3-chloro-2,4-difluorophenyl)acetyl]-2,3-dihydro-1H-indol-5-yl}-7-methyl-7H-pyrrolo[2,3-d]pyrimidin-4-amine;
5-(1-{[3-fluoro-5-(trifluoromethyl)phenyl]acetyl}-2,3-dihydro-1H-indol-5-yl)-7-methyl-7H-pyrrolo[2,3-d]pyrimidin-4-amine;
7-[(methyloxy)methyl]-5-{1-[(3-methylphenyl)acetyl]-2,3-dihydro-1H-indol-5-yl}-7H-pyrrolo[2,3-d]pyrimidin-4-amine;
7-methyl-5-{1-[(1-methyl-1H-pyrrol-2-yl)acetyl]-2,3-dihydro-1H-indol-5-yl}-7H-pyrrolo[2,3-d]pyrimidin-4-amine;
5-{1-[(2,5-difluorophenyl)acetyl]-2,3-dihydro-1H-indol-5-yl}-7-(1-methylethyl)-7H-pyrrolo[2,3-d]pyrimidin-4-amine;
5-{1-[(5-chloro-2-fluorophenyl)acetyl]-2,3-dihydro-1H-indol-5-yl}-7-methyl-7H-pyrrolo[2,3-d]pyrimidin-4-amine;
5-{1-[(2,5-difluorophenyl)acetyl]-2,3-dihydro-1H-indol-5-yl}-7-[2-(4-morpholinyl)ethyl]-7H-pyrrolo[2,3-d]pyrimidin-4-amine;
5-{1-[(2,4-difluorophenyl)acetyl]-2,3-dihydro-1H-indol-5-yl}-7-methyl-7H-pyrrolo[2,3-d]pyrimidin-4-amine;
5-{1-[(3,4-difluorophenyl)acetyl]-2,3-dihydro-1H-indol-5-yl}-7-methyl-7H-pyrrolo[2,3-d]pyrimidin-4-amine;
phenylmethyl[2-(4-amino-3-{1-[(2,5-difluorophenyl)acetyl]-2,3-dihydro-1H-indol-5-yl}furo[3,2-c]pyridin-7-yl)ethyl]carbamate;
5-{1-[(2,5-difluorophenyl)acetyl]-2,3-dihydro-1H-indol-5-yl}-7-(3-methylbutyl)-7H-pyrrolo[2,3-d]pyrimidin-4-amine;
5-{1-[(2,5-difluorophenyl)acetyl]-2,3-dihydro-1H-indol-5-yl}-7-[2-(dimethylamino)ethyl]-7H-pyrrolo[2,3-d]pyrimidin-4-amine;
5-{1-[(6-chloro-2-pyridinyl)acetyl]-2,3-dihydro-1H-indol-5-yl}-7-methyl-7H-pyrrolo[2,3-d]pyrimidin-4-amine;
3-{1-[(3-chloro-2,4-difluorophenyl)acetyl]-2,3-dihydro-1H-indol-5-yl}-1-methyl-1H-pyrazolo[3,4-d]pyrimidin-4-amine;
7-(2-aminoethyl)-3-{1-[(2,5-difluorophenyl)acetyl]-2,3-dihydro-1H-indol-5-yl}furo[3,2-c]pyridin-4-amine;
4-amino-3-{1-[(2,5-difluorophenyl)acetyl]-2,3-dihydro-1H-indol-5-yl}furo[3,2-c]pyridine-7-carbonitrile;
5-{1-[(3,5-dimethyl-1H-pyrazol-1-yl)acetyl]-2,3-dihydro-1H-indol-5-yl}-7-methyl-7H-pyrrolo[2,3-d]pyrimidin-4-amine;
5-[4-fluoro-1-(phenylacetyl)-2,3-dihydro-1H-indol-5-yl]-7-methyl-7H-pyrrolo[2,3-d]pyrimidin-4-amine;
5-{4-fluoro-1-[(1-methyl-1H-pyrrol-2-yl)acetyl]-2,3-dihydro-1H-indol-5-yl}-7-methyl-7H-pyrrolo[2,3-d]pyrimidin-4-amine;
5-{1-[(2,5-difluorophenyl)acetyl]-4-fluoro-2,3-dihydro-1,4-indol-5-yl}-7-methyl-7H-pyrrolo[2,3-d]pyrimidin-4-amine;
5-{1-[(2,5-difluorophenyl)acetyl]-2,3-dihydro-1H-indol-5-yl}furo[2,3-d]pyrimidin-4-amine;
5-(1-{[3-(trifluoromethyl)phenyl]acetyl}-2,3-dihydro-1H-indol-5-yl)furo[2,3-d]pyrimidin-4-amine;
5-{1-[(3-chloro-5-fluorophenyl)acetyl]-2,3-dihydro-1H-indol-5-yl}furo[2,3-d]pyrimidin-4-amine;
5-{1-[(3-methylphenyl)acetyl]-2,3-dihydro-1H-indol-5-yl}furo[2,3-d]pyrimidin-4-amine;
5-(1-{[3-fluoro-5-(trifluoromethyl)phenyl]acetyl}-2,3-dihydro-1H-indol-5-yl)furo[2,3-d]pyrimidin-4-amine;
5-{1-[(2,5-difluorophenyl)acetyl]-2,3-dihydro-1H-indol-5-yl}-7-[2-(4-piperidinyl)ethyl]-7H-pyrrolo[2,3-d]pyrimidin-4-amine;
7-methyl-5-{1-[(6-methyl-2-pyridinyl)acetyl]-2,3-dihydro-1H-indol-5-yl}-7H-pyrrolo[2,3-d]pyrimidin-4-amine;
5-(1-{[4-fluoro-3-(trifluoromethyl)phenyl]acetyl}-2,3-dihydro-1H-indol-5-yl)-7-methyl-7H-pyrrolo[2,3-d]pyrimidin-4-amine;
5-{1-[(2,5-difluorophenyl)acetyl]-2,3-dihydro-1H-indol-5-yl}-7-(3-oxetanyl)-7H-pyrrolo[2,3-d]pyrimidin-4-amine;
3-{1-[(2,5-difluorophenyl)acetyl]-2,3-dihydro-1H-indol-5-yl}-7-[2-(dimethylamino)ethyl]furo[3,2-c]pyridin-4-amine;
7-methyl-5-(1-{[6-(trifluoromethyl)-2-pyridinyl]acetyl}-2,3-dihydro-1H-indol-5-yl)-7H-pyrrolo[2,3-d]pyrimidin-4-amine;
7-(3-oxetanyl)-5-(1-{[3-(trifluoromethyl)phenyl]acetyl}-2,3-dihydro-1H-indol-5-yl)-7H-pyrrolo[2,3-d]pyrimidin-4-amine;
7-[2-(4-morpholinyl)ethyl]-5-(1-{[3-(trifluoromethyl)phenyl]acetyl}-2,3-dihydro-1H-indol-5-yl)-7H-pyrrolo[2,3-d]pyrimidin-4-amine;
7-(1-methylethyl)-5-(1-{[3-(trifluoromethyl)phenyl]acetyl}-2,3-dihydro-1H-indol-5-yl)-7H-pyrrolo[2,3-d]pyrimidin-4-amine;
7-(3-methylbutyl)-5-(1-{[3-(trifluoromethyl)phenyl]acetyl}-2,3-dihydro-1H-indol-5-yl)-7H-pyrrolo[2,3-d]pyrimidin-4-amine;
4-{1-[(3-methylphenyl)acetyl]-2,3-dihydro-1H-indol-5-yl}-1H-pyrazolo[3,4-c]pyridin-3-amine;
7-chloro-3-{1-[(2,5-difluorophenyl)acetyl]-2,3-dihydro-1H-indol-5-yl}furo[3,2-c]pyridin-4-amine;
7-(3-azetidinyl)-5-(1-{[3-(trifluoromethyl)phenyl]acetyl}-2,3-dihydro-1H-indol-5-yl)-7H-pyrrolo[2,3-d]pyrimidin-4-amine;
7-(1-methyl-3-azetidinyl)-5-(1-{[3-(trifluoromethyl)phenyl]acetyl}-2,3-dihydro-1H-indol-5-yl)-7H-pyrrolo[2,3-d]pyrimidin-4-amine;
7-[2-(dimethylamino)ethyl]-5-(1-{[3-(trifluoromethyl)phenyl]acetyl}-2,3-dihydro-1H-indol-5-yl)-7H-pyrrolo[2,3-d]pyrimidin-4-amine;
5-(4-fluoro-1-{[3-(trifluoromethyl)phenyl]acetyl}-2,3-dihydro-1H-indol-5-yl)-7-methyl-7H-pyrrolo[2,3-d]pyrimidin-4-amine;
5-{4-fluoro-1-[(6-methyl-2-pyridinyl)acetyl]-2,3-dihydro-1H-indol-5-yl}-7-methyl-7H-pyrrolo[2,3-d]pyrimidin-4-amine;
5-(4-fluoro-1-{[6-(trifluoromethyl)-2-pyridinyl]acetyl}-2,3-dihydro-1H-indol-5-yl)-7-methyl-7H-pyrrolo[2,3-d]pyrimidin-4-amine;
5-{1-[(3,5-dimethyl-1H-pyrazol-1-yl)acetyl]-4-fluoro-2,3-dihydro-1H-indol-5-yl}-7-methyl-7H-pyrrolo[2,3-d]pyrimidin-4-amine;
5-(4-fluoro-1-{[4-fluoro-3-(trifluoromethyl)phenyl]acetyl}-2,3-dihydro-1H-indol-5-yl)-7-methyl-7H-pyrrolo[2,3-d]pyrimidin-4-amine;
3-{1-[(2,5-difluorophenyl)acetyl]-4-fluoro-2,3-dihydro-1H-indol-5-yl}furo[3,2-c]pyridin-4-amine;
5-{4-fluoro-1-[(4-fluorophenyl)acetyl]-2,3-dihydro-1H-indol-5-yl}-7-methyl-7H-pyrrolo[2,3-d]pyrimidin-4-amine;
4-(1-[3-(trifluoromethyl)phenyl)acetyl]-2,3-dihydro-1H-indol-5-yl)-1H-pyrazolo[3,4-c]pyridin-3-amine;
1-methyl-4-(1-{[3-(trifluoromethyl)phenyl]acetyl}-2,3-dihydro-1H-indol-5-yl)-1H-pyrazolo[3,4-c]pyridin-3-amine;
7-(3-azetidinyl)-5-{1-[(2,5-difluorophenyl)acetyl]-2,3-dihydro-1H-indol-5-yl}-7H-pyrrolo[2,3-d]pyrimidin-4-amine;
7-[2-(4-piperidinyl)ethyl]-5-(1-{[3-(trifluoromethyl)phenyl]acetyl}-2,3-dihydro-1H-indol-5-yl)-7H-pyrrolo[2,3-d]pyrimidin-4-amine;
7-(2-aminoethyl)-3-{1-[(2,5-difluorophenyl)acetyl]-4-fluoro-2,3-dihydro-1H-indol-5-yl}furo[3,2-c]pyridin-4-amine;
3-{1-[(3,5-dimethyl-1H-pyrazol-1-yl)acetyl]-2,3-dihydro-1H-indol-5-yl}-1-methyl-1H-pyrazolo[3,4-d]pyrimidin-4-amine;
5-(1-{[3-(trifluoromethyl)phenyl]acetyl}-2,3-dihydro-1H-indol-5-yl)-1H-pyrrolo[2,3-d]pyrimidin-4-amine;
5-{4-chloro-1-[(6-methyl-2-pyridinyl)acetyl]-2,3-dihydro-1H-indol-5-yl}-7-methyl-7H-pyrrolo[2,3-d]pyrimidin-4-amine; and
5-(4-chloro-1-{[6-(trifluoromethyl)-2-pyridinyl]acetyl}-2,3-dihydro-1H-indol-5-yl)-7-methyl-7H-pyrrolo[2,3-d]pyrimidin-4-amine;
   and salts thereof, including pharmaceutically acceptable salts thereof.

In one embodiment, the inhibitor is the organic molecule: 5-bromo-N₄-2-pyridinyl-N₂-[3-(1,2,3,6-tetrahydro-4-pyridinyl)-1H-indol-5-yl]-2,4-pyrimidinediamine. This organic molecule is a small molecule inhibitor of PERK protein. While PERK inhibitors are known in the art, known PERK inhibitors have not been used in the FOXO pathway. The present inventors have now surprisingly found that this organic molecule blocks the potentiating effects of PERK on FOXO. One normal function of PERK protein is to protect secretory cells from endoplasmic reticulum (ER) stress. It has been shown that an absence of PERK during embryonic development causes Type 1 diabetes due to loss of pancreatic islet cells. Further, loss of ER homeostasis and an accumulation of misfolded proteins can contribute to cardiovascular and degenerative diseases. PERK also responds to unfolded proteins in a cell. ER stress due to unfolded proteins is elevated in cancers and inhibition of the ER stress response can limit tumor growth. However, there has been no link until now to show that inhibition of PERK can treat or prevent insulin resistance syndrome by targeting the FOXO pathway.

The disclosed methods may further comprise administration of a peroxisome proliferator-activated receptor (PPAR) agonist and/or an anti-diabetic agent. The PPAR agonist may include, but is not limited to, PPAR-alpha agonists, PPAR-beta agonists, PPAR-gamma agonists, and PPAR-delta agonists. In one embodiment, the PPAR agonist is selected from the group consisting of mono-PPAR-alpha agonists, mono-PPAR-beta agonists, mono-PPAR-gamma agonists, mono-PPAR-delta agonists, dual PPAR-alpha and gamma agonists, N-[(4-methoxyphenoxy)carbonyl]-N-{4-[2-(5-methyl-2-phenyl-1,3-oxazol-4-yl)ethoxy]benzyl}glycine (Muraglitazar), 2-[(4-methoxyphenoxy)carbonyl-[(1S)-1-[4-[2-(5-methyl-2-phenyl-1,3-oxazol-4-yl)ethoxy]phenyl]ethyl]amino]acetic acid (peliglitazar), N-(o-benzoylphenyl)-O-[2-(5-methyl-2-phenyl-4-oxazolypethyl]-L-tyrosine (Farglitazar), thiazolidinediones class of PPAR-agonists, (RS)-5-(4-[(6-hydroxy-2,5,7,8-tetramethylchroman-2-yl)methoxy]benzyl)thiazolidine-2,4-dione (Troglitazone), (RS)-5-(4-[2-(5-ethylpyridin-2-yl)ethoxy]benzyl)thiazolidine-2,4-dione (Pioglitazone), (RS)-5-[4-(2-[methyl (pyridin-2-yl)amino]ethoxy)benzyl]thiazolidine-2,4-di one (Rosiglitazone), 5-[[6-[(2-fluorophenyl)methoxy]naphthalen-2-yl]methyl]-1,3-thiazolidine-2,4-dione (netoglitazone or MCC555), 5-[(2,4-Dioxo-1,3-thiazolidin-5-yl)methyl]-2-methoxy-N-[4-(trifluoromethyl)benzyl)benzamide (KRP 297), 4-[[4-[2-(5-methyl-2-phenyl-1,3-oxazol-4-yl)ethoxy]phenyl]methyl]-1,2-oxazolidine-3,5-dione (JTT-501), 12-(4-Chlorophenyl)-2,2-dichlorododecanoic acid (BM 17.0744), L764486, 2-[2-methyl-4-([4-methyl-2-[4-(trifluoromethyl)phenyl)thiazol-5-yl)methylthio)phenoxy)acetic acid (GW501516), (25)-2-ethoxy-3-[4-(2-phenoxazin-10-ylethoxy)phenyl]propanoic acid (NN622), 2-(4-{2-[(4-chlorobenzoyl)amino]ethyl}phenoxy)-2-methylpropanoic acid (bezafibrate), 5-(2,5-dimethylphenoxy)-2,2-dimethyl-pentanoic acid (gemfibrozil), fibrate class of PPAR-agonists, (S)-3-[4-[2-(Phenoxazin-10-yl)ethoxy]phenyl]-2-ethoxypropanoicacid (DRF 2725), [[4-chloro-6-[(2,3-dimethylphenyl)amino]-2-pyrimidinyl]thio]-acetic acid (WY 14,643), 3-{4-[2-(Benzooxazol-2-yl-methyl-amino)-ethoxy]-phenyl}-2-ethoxy-propionic acid (SB 213068), (2S)-2-Ethoxy-3-[4-[2-(4-methylsulfonyloxyphenyl)ethoxy]phenyl]propanoic acid (Tesaglitazar; AZ 242), (±)-5-[[4-[2-(methyl-2-pyridinylamino)ethoxy]phenyl]methyl]-2,4-thiazolidinedione, (Z)-2-butenedioate and 1-[[p-[2-(3-ethyl-4-methyl-2-oxo-3-pyrroline-1-carboxamido)ethyl]phenyl]sulfonyl]-3-(trans-4-methylcyclohexyl)urea (Avandaryl), 2-methoxy-3-[4-[3-[4-(phenoxy)phenoxy]propoxy]phenyl]propanoic acid (Naveglitazar), (2S)-3-[4-[2-(4a,10a-dihydrophenoxazin-10-yl)ethoxy]phenyl]-2-ethoxypropanoic acid (Ragaglitazar; NN622), PLX 204, PLX 134, PLX 203, 5-(4-((6-(4-amino-3,5-dimethylphenoxy)-1-methyl-1H-benzo[d]imidazol-2-yl)methoxy)benzypthiazolidine-2,4-dione (CS 7017), DRF 10945, AVE 0847, AVE 8134, 641597 (GSK), 590735 (GSK), MK 767, AA 10090, LY 674, LY 929, T 131, DRF 4158, CLX 0921, NS 220, LY 293111, DRF 4832, GW 7282, 501516 (GSK), LG 100754, GW 544, AR H049020, AK-109, E-3030 (Eisai), CS-7017 (Sankyo), DRF-10945, KRP-101, ONO-5129, TY-51501, GSK-677954, LSN-862, LY-518674, GW-590735, KT6-207, K-111 (Roche), Bay-54-9801 (GSK), R-483 (Roche), EMD-336340 (Merck KGaA), LR-90 (Merck KGaA), CLX-0940, CLX-0921, LG-100754, GW-409890, SB-219994, NIP-223, T-174 (Tanabe Seiyaku), (+/−)-5-(p-((3,4-Dihydro-3-methyl-4-oxo-2-quinazolinyl)methoxy)benzyl)-2,4-thiazolidinedione (balaglitazone; DRF-2593), VDO-52, GW-1929, NC-2100, 5-[[6-[(2-fluorophenyl)methoxy]naphthalen-2-yl]methyl]-1,3-thiazolidine-2,4-dione (netoglitazone), 5-{4-[(1-methylcyclohexyl)methoxy]benzyl}-1,3-thiazolidine-2,4-dione (ciglitazone), LGD 1268, LG 101506, LGD 1324, GW 9578, 5-[(2-benzyl-3,4-dihydro-2H-chromen-6-yl)methyl]-1,3-thiazolidine-2,4-dione (Englitazone), and 5-[(4-[3-(5-methyl-2-phenyl-1,3-oxazol-4-yl)propanoyl]phenyl)methyl]-1,3-thiazolidine-2,4-dione (Darglitazone).

The disclosed methods may further comprise administration of an agent used in the treatment of diabetes, obesity or insulin resistance syndrome.

As the insulin signaling pathway is conserved throughout the animal kingdom, the present inventors have now found that the endoplasmic reticulum (ER) stress pathway in Drosophila is an inducer of FOXO activity. Whereas, in mammals, ER stress effects three pathways: activating transcription factor 6 (ATF6), IRE1 and PERK (which in turn effects the FOXO pathway). ATF6 is an ER stress-regulated transmembrane transcription factor that activates the transcription of ER molecules. IRE1 is a ser/thr protein kinase that possesses endonuclease activity and is important in altering gene expression as a response to ER stress signals. Accordingly, as ER stress induces the nuclear localization and activity of FOXO, modulators of ER stress would be useful in decreasing the effects of ER stress on the activity of FOXO.

The agent used in the treatment of diabetes, obesity or insulin resistance syndrome may include, but is not limited to, ER stress modulators such as PBA (4-phenyl butyrate), TUDCA (tauroursodeoxycholic acid), TMAO (trimethylamine N-oxide), and derivatives thereof; anti-diabetic agents, anti-obesity agents, anti-dyslipidemia agent or anti-atherosclerosis agent, aspirin, vitamins, minerals, and anti-hypertensive agents.

Anti-diabetic agent includes, but is not limited to, insulin or hypoglycemic agents. Hypoglycemic agents include, but are not limited to, oral hypoglycemic agents such as sulfonylureas, tolbutamide, metformin, chlorpropamide, acetohexamide, tolazamide, glyburide, etc. Anti-obesity agent includes, but is not limited to, appetite suppressants. Anti-dyslipidemia agent or anti-atherosclerosis agent includes, but is not limited to, cholesterol lowering agents. Cholesterol lowering agents include, but are not limited to, HMg-CoA and reductase inhibitors such as lovastatin, atorvastatin, simvastatin, pravastatin, fluvastatin, etc.

It has been shown that ER stress can also be induced by genetic models of obesity such as the ob/ob mouse as well as diet-induced obesity. Use of chemical chaperones to overcome ER stress has been reported to limit activation of the three pathways, to decrease insulin and glucose levels and to improve glucose tolerance and insulin tolerance test results in both insulin resistant ob/ob mice and in obese diabetic humans.

Accordingly, the anti-diabetic agent may be used in combination with a chemical chaperon. The chemical chaperon may include, but is not limited to, imidodicarbonimidic diamide (biguanides), sulfonylureas, insulin and analogs thereof, peroxisome proliferator-activated receptor-gamma agonists, dual PPAR agonists and PPAR pan agonists, combination therapies, meglitinides, alpha-glucosidase inhibitors, glucagon-like peptide-1 (GLP-1) analogues and agonists, dipeptidyl peptidase IV (DPP-IV) inhibitors, pancreatic lipase inhibitors, sodium glucose co-transporter (SGLT) inhibitors, and amylin analog.

An example of biguanides is metformin. Examples of sulfonylureas include glimepiride, glyburide, glibenclamide, glipizide and gliclazide. Analogs of insulin include insulin lispro, insulin glargine, exubera, AERx insulin diabetes management system, AIR inhaled insulin, oralin, insulin detemir and insulin glulisine. Peroxisome proliferator-activated receptor-gamma agonists include rosiglitazone, pioglitazone, isaglitazone, rivoglitazone, T-131, MBX-102 and R-483 CLX-0921. Dual PPAR agonists and PPAR pan agonists include BMS-398585, tesaglitazar, muraglitazar, naveglitazar, TAK-559, netoglitazone, GW-677594, AVE-0847, LY-929 and ONO-5129. Combination therapies include metformin/glyburide, metformin/rosiglitazone, metformin and glipizide. Meglitinides include repaglinide or nateglinide. Alpha-glucosidase inhibitors include acarbose, miglitol and voglibose. GLP-1 analogues and agonists include Exenatide, Exenatide LAR, Liraglutide, CJC-1131, AVE-0010, BIM-51077, NN-2501 and SUN-E7001. DPP-IV inhibitors include LAF-237, MK-431 (Merck and Co), dipeptidyl peptidase IV (PSN-9301; Probiodrug Prosidion), 815541 (GlaxoSmithKline-Tanabe), 823093 (GlaxoSmithKline), 825964 (GlaxoSmithKline) and (1S,3S,5S)-2-[(2S)-2-amino-2-(3-hydroxy-1-adamantyl)acetyl]-2-azabicyclo[3.1.0]hexane-3-carbonitrile (BMS-477118). An example of pancreatic lipase inhibitors is orlistat. SGLT inhibitors include T-1095 (Tanabe-J&J), AVE-2268 and 2-(4-methoxybenzyl)phenyl 6-O-(ethoxycarbonyl)-β-D-glucopyranoside (869682; GlaxoSmithKline-Kissei). An example of amylin analog is pramlintide.

The disclosed chemical chaperone or the disclosed ER stress modulator may be used in combination with an anti-obesity agent. The anti-obesity agent may include, but is not limited to, pancreatic lipase inhibitors, serotonin and norepinephrine reuptake inhibitors, noradrenergic anorectic agents, peripherally acting agents, centrally acting agents, thermogenic agents, cannabinoid CB1 antagonists, cholecystokinin (CCK) agonists, lipid metabolism modulator, glucagon-like peptide 1 agonist, leptin agonist, beta-3 adrenergic agonists, peptide hormone, CNS modulator, neurotrophic factor, 5HT2C serotonin receptor agonist, methamphetamine HCl, 1426 (Sanofi-Aventis), 1954 (Sanofi-Aventis), c-2624 (Merck & Co), c-5093 (Merck & Co), and T71 (Tularik).

As previously mentioned, pancreatic lipase inhibitors may include orlistat. An example of serotonin and norepinephrine reuptake inhibitors is sibutramine. Noradrenergic anorectic agents include phentermine and mazindol. Peripherally acting agents include 2-(Hexadecyloxy)-6-methyl-4H-3,1-benzoxazin-4-one (ATL-962; Alizyme), HMR-1426 (Aventis) and G1-181771 (GlaxoSmithKline). Centrally acting agents include Recombinant human ciliary neurotrophic factor, 5-(4-Chlorophenyl)-1-(2,4-dichloro-phenyl)-4-methyl-N-(piperidin-1-yl)-1H-pyrazole-3-carboxamide (Rimonabant (SR-141716; Sanofi-Synthelabo)), BVT-933 (GlaxoSmithKline/Biovitrum), (±)-2-(tert-Butylamino)-1-(3-chlorophenyl)propan-1-one (Bupropion SR; GlaxoSmithKline) and P-57 (Phytopharm). An example of thermogenic agents is TAK-677 (AJ-9677) (Dainippon/Takeda). Cannabinoid CB1 antagonists include acomplia and SLV319. An example of CCK agonists is GI 181771 (GSK). An example of lipid metabolism modulator is AOD₉₆₀₄ (Monash University/Metabolic Pharmaceuticals). An example of glucagon-like peptide 1 agonist is AC137 (Amylin). An example of leptin agonist is second generation leptin (Amgen). Beta-3 adrenergic agonists include (7S)-5,6,7,8-Tetrahydro-7-[[(R)-2-hydroxy-2-(3-chlorophenyl)ethyl]amino]-2-[(ethoxycarbonyl)methoxy]naphthalene (SR58611; Sanofi-Aventis), CP 331684 (Pfizer), LY 377604 (Eli Lilly) and n5984 (Nisshin Kyorin Pharmaceutical). An example of peptide hormone is peptide YY [3-36] (Nastech). An example of CNS modulator is S2367 (Shionogi & Co. Ltd.). An example of neurotrophic factor is peg axokine. An example of 5HT2C serotonin receptor agonist is APD356.

The disclosed chemical chaperone or the disclosed ER stress modulator may be used in combination with an anti-dyslipidemia agent or anti-atherosclerosis agent. The anti-dyslipidemia agent or anti-atherosclerosis agent may include, but is not limited to, HMG-CoA reductase inhibitors, fibrates, bile acid sequestrants, niacin (immediate and extended release), anti-platelets, angiotensin-converting enzyme (ACE) inhibitors, angiotensin II receptor antagonists, acyl-CoA cholesterol acetyltransferase (ACAT) inhibitors, cholesterol absorption inhibitors, nicotinic acid derivatives, cholesterol ester transfer protein (CETP) inhibitors, microsomal triglyceride transfer protein (MTTP) inhibitors, other cholesterol modulators, bile acid modulators, peroxisome proliferation activated receptor (PPAR) agonists, gene-based therapies, composite vascular protectant, 4,6-Di-tert-butyl-2,3-dihydro-2,2-dipentyl-5-benzofuranol (BO-653; Chugai), glycoprotein inhibitors, aspirin and analogs thereof, combination therapies, ileal bile acid transporter (IBAT) inhibitors, squalene synthase inhibitors, monocyte chemoattractant protein (MCP-1) inhibitors, liver X receptor agonists, and other new approaches.

HMG-CoA reductase inhibitors include atorvastatin, pravastatin, simvastatin, lovastatin, fluvastatin, cerivastatina, rosuvastatin and pitivastatin. Fibrates include ciprofibrate, bezafibrate, clofibrate, fenofibrate and gemfibrozil. Bile acid sequestrants include cholestyramine, colestipol and colesevelam. Anti-platelets include aspirin, clopidogrel and ticlopidine. ACE inhibitors include ramipril and enalapril. Angiotensin II receptor antagonists include losartan and potassium. ACAT inhibitors include avasimibe, eflucimibe, CS-505 (Sankyo and Kyoto), SMP-797 (Sumito). Cholesterol absorption inhibitors include ezetimibe and pamaqueside. An example of nicotinic acid derivatives is nicotinic acid. CETP inhibitors include CP-529414 (Pfizer), JTT-705 (Japan Tobacco), CETi-1 and torcetrapib. MTTP inhibitors include implitapide, R-103757 and CP-346086 (Pfizer). Other cholesterol modulators include NO-1886 (Otsuka/TAP Pharmaceutical), CI-1027 (Pfizer) and WAY-135433 (Wyeth-Ayerst). Bile acid modulators include GT102-279 (GelTex/Sankyo), HBS-107 (Hisamitsu/Banyu), BTG-511 (British Technology Group), BARI-1453 (Aventis), S-8921 (Shionogi), SD-5613 (Pfizer) and AZD-7806 (AstraZeneca). PPAR agonists include Tesaglitazar (AZ-242) (AstraZeneca), Netoglitazone (MCC-555) (Mitsubishi/Johnson & Johnson), (2S)-3-[4-[2-(5-methyl-2-phenyl-1,3-oxazol-4-yl)ethoxy]phenyl]-2-[(4-oxo-4-phenylbutan-2-yl)amino]propanoic acid (GW-409544; Ligand Pharmaceutical s/Glaxo SmithKline), 2-[2-methyl-4-([4-methyl-2-[4-(trifluoromethyl)phenyl)thiazol-5-yl)methylthio)phenoxy)acetic acid (GW-501516; Ligand Pharmaceuticals/GlaxoSmithKline), LY-929 (Ligand Pharmaceuticals and Eli Lilly), 2-methyl-2-[4-[2-[5-methyl-2-(4-phenylphenyl)-1,3-oxazol-4-yl]ethoxy]phenoxy]propanoic acid (LY-465608; Ligand Pharmaceuticals and Eli Lilly), 2-methyl-2-[4-[3-[1-[(4-methylphenyl)methyl]-5-oxo-2H-1,2,4-triazol-3-yl]propyl]phenoxy]propanoic acid (LY-518674; Ligand Pharmaceuticals and Eli Lilly) and MK-767 (Merck and Kyorin). Gene-based therapies include AdGVVEGF121.10 (GenVec), ApoA1 (UCB Pharma/Groupe Fournier), EG-004 (Trinam) (Ark Therapeutics) and ATP-binding cassette transporter-A1 (ABCA1) (CV Therapeutics/Incyte, Aventis, Xenon). An example of composite vascular protectant is AGI-1067 (Atherogenics). Glycoprotein IIb/IIIa inhibitors include Roxifiban (Bristol-Myers Squibb), Gantofiban (Yamanouchi) and Cromafiban (Millennium Pharmaceuticals). Aspirin and analogs thereof include asacard, slow-release aspirin and pamicogrel. Combination therapies include niacin/lovastatin, amlodipine/atorvastatin and simvastatin/ezetimibe. An example of IBAT inhibitors is S-89-21 (Shionogi). Squalene synthase inhibitors include BMS-188494 I (Bristol-Myers Squibb), CP-210172 (Pfizer), CP-295697 (Pfizer), CP-294838 (Pfizer) and TAK-475 (Takeda). MCP-1 inhibitors include RS-504393 (Roche Bioscience) and other MCP-1 inhibitors (GlaxoSmithKline, Teijin, and Bristol-Myers Squibb). Liver X receptor agonists include GW-3965 (GlaxoSmithKline) and TU-0901317 (Tularik). Other new approaches include MBX-102 (Metabolex), NO-1886 (Otsuka) and Gemcabene (Pfizer).

The disclosed chemical chaperone or the disclosed ER stress modulator may be used in combination with an anti-hypertension agent. The anti-hypertension agent may include, but is not limited to, diuretics, alpha-blockers, beta-blockers, Ca²⁺ channel blockers, angiotensin converting enzyme (ACE) inhibitors, angiotensin II (AT-II) antagonists, vasopeptidase inhibitors, dual neutral endopeptidase and enotheline converting enzyme (NEP/ECE) inhibitors, angiotensin vaccines, ACE/NEP inhibitors, Na⁺/K⁺ ATPase modulators, vasodilators, naturetic peptides, angiotensin receptor blockers (ARBs), ACE crosslink breakers, endothelin receptor agonists, combination therapies, and MC4232 (University of Manitoba/Medicure).

Diuretics include chlorthalidone, metolazone, indapamide, bumetanide, ethacrynic acid, furosemide, torsemide, amiloride HCl, spironolactone and triamterene. Alpha-blockers include doxazosin mesylate, prazosin HCl and terazosin HCl. Beta-blockers include acebutolol, atenolol, betaxolol, bisoprolol fumarate, carteolol HCl, metoprolol tartrate, metoprolol succinate, nadolol, penbutolol sulfate, pindolol, propanolol HCl, timolol maleate and carvedilol. Ca²⁺ channel blockers include amlodipine besylate, felodipine, isradipine, nicardipine, nifedipine, nisoldipine, diltiazem HCl, verapamil HCl, azelnidipine, pranidipine, graded diltiazem formulation, (s)-amlodipine and clevidipine. ACE inhibitors include benazepril hydrochloride, captopril, enalapril maleate, fosinopril sodium, lisinopril, moexipril, perindopril, quinapril hydrochloride, ramipril and trandolapril. AT-II antagonists include losartan, valsartan, irbesartan, candesartan, telmisartan, eprosartan, olmesarta and YM-358 (Yamanouchi). Vasopeptidase inhibitors include omapatrilat, gemopatrilat, fasidotril, sampatrilat, AVE 7688 (Aventis), M100240 (Aventis), Z13752A (Zambon/GSK) and 796406 (Zambon/GSK). NEP/ECE inhibitors include SLV306 (Solvay), NEP inhibitors such as ecadotril, aldosterone antagonists such as eplerenone, renin inhibitors such as Aliskiren (Novartis), SPP 500 (Roche/Speedel), SPP600 (Speedel) and SPP 800 (Locus/Speedel). An example of angiotensin vaccines is PMD-3117 (Protherics). ACE/NEP inhibitors include AVE-7688 (Aventis) and GW-660511 (Zambon SpA). Na⁺/K⁺ ATPase modulators include PST-2238 (Prassis-Sigma-Tau) and endothelin antagonists such as PD-156707 (Pfizer). Vasodilators include NCX-4016 (NicOx) and LP-805 (Pola/Wyeth). An example of naturetic peptides is BDNP (Mayo Foundation). An example of ARBs is pratosartan. An example of ACE crosslink breakers is alagebrium chloride. Endothelin receptor agonists include tezosentan (Genentech), ambrisentan (Myogen), BMS 193884 (BMS), sitaxsentan (Encysive Pharmaceuticals), SPP301 (Roche/Speedel), Darusentan (Myogen/Abbott), J104132 (BanyulMerck & Co.), TBC3711 (Encysive Pharmaceuticals) and SB 234551 (GSK/Shionogi). Combination therapies include benazepril hydrochloride/hydrochlorothiazide, captopril/hydrochlorothiazide, enalapril maleate/hydrochlorothiazide, lisinopril/hydrochlorothiazide, losartan/hydrochlorothiazide, atenolol/chlorthalidone, bisoprolol fumarate/hydrochlorothiazide, metoprolol tartrate/hydrochlorothiazide, amlodipine besylatelbenazepril hydrochloride, felodipine/enalapril maleate, verapamil hydrochloride/trandolapril, lercanidipine and enalapril, olmesartan/hydrochlorothiazide, eprosartanlhydrochlorothiazide, amlodipine besylate/atorvastatin and nitrendipine/enalapril.

The disclosed chemical chaperone or the disclosed ER stress modulator may be used in combination with vitamin, mineral, and other nutritional supplements.

For the avoidance of doubt, the disclosed chemical chaperone may be used in any combination thereof as disclosed above. The disclosed ER stress modulator may also be used in any combination thereof as disclosed above.

In some embodiments, there is further provided the use of an inhibitor of PERK gene, or a functional variant thereof, or an inhibitor of the activity of PERK protein, or a functional variant thereof, in the manufacture of a medicament for the treatment or prevention of insulin resistance syndrome.

The term “preventing” refers to a method of barring the organism from acquiring the abnormal condition, while the term “treating” refers to a method of alleviating or abrogating the abnormal condition in the organism.

In other embodiments, there is also provided the use of an inhibitor of PERK gene, or a functional variant thereof, or an inhibitor of the activity of PERK protein or a functional variant thereof, in the manufacture of a medicament to reduce activity of transcription factors of the FOXO family (Foxo1, 3a, 4 and 6) for the treatment or prevention of insulin resistance syndrome.

The disclosed inhibitor of PERK gene or a functional variant thereof, or an inhibitor of PERK protein, or a functional variant thereof, may be used in a pharmaceutical composition. Accordingly, in one embodiment, there is provided a pharmaceutical composition comprising an inhibitor of PERK gene or a functional variant thereof, or an inhibitor of PERK protein or a functional variant thereof.

The pharmaceutical composition may further comprise a pharmaceutically acceptable carrier and/or diluent and/or excipient.

As used herein, the terms “administering” or “administration”, or grammatical variants thereof, refer to the delivery of the disclosed inhibitor alone or in combination with the compounds disclosed herein to an organism for the purpose of prevention or treatment of insulin resistance syndrome.

As used herein, the term “pharmaceutically acceptable carrier” refers to media generally accepted in the art for the delivery of biologically active agents, i.e. the disclosed inhibitor alone or in combination with any of the disclosed compounds in the context of the specification, to mammals, e.g. humans. Such carriers are generally formulated according to a number of factors well within the purview of those of ordinary skill in the art to determine and account for. These include, without limitation, the type and nature of the active agent, being formulated; the subject to which the agent-containing composition is to be administered; the intended route of administration of the composition; and the therapeutic indication being targeted. Pharmaceutically acceptable carriers include both aqueous and non-aqueous liquid media, as well as a variety of solid and semi-solid dosage forms. Such carriers can include a number of different ingredients and additives in addition to the active agent, such additional ingredients being included in the formulation for a variety of reasons, e.g., stabilization of the active agent, well known to those of ordinary skill in the art. Non-limiting examples of a pharmaceutically acceptable carrier are hyaluronic acid and salts thereof, and microspheres (including, but not limited to, poly(D,L)-lactide-co-glycolic acid copolymer (PLGA), poly(L-lactic acid) (PLA), poly(caprolactone) (PCL) and bovine serum albumin (BSA)).

The term “diluent” generally refers to a substance that usually makes up the major portion of the pharmaceutical composition. Suitable diluents include sugars such as lactose, sucrose, mannitol, and sorbitol; starches derived from wheat, corn rice, and potato; and celluloses such as microcrystalline cellulose. The amount of diluent in the composition can range from about 10% to about 90% by weight of the total composition, or from about 20% to about 90% by weight, or from about 30% to about 90% by weight, or from about 10% to about 80% by weight, or from about 20% to about 80% by weight, or from about 30% to about 80% by weight.

The term “excipient” refers to a pharmaceutically acceptable additive, other than the active ingredient, included in a formulation and having different purposes depending, for example, on the nature of the drug, and the mode of administration. Examples of excipients include, without limitation, carriers, co-solvents, stabilizing agents, solubilizing agents and surfactants, buffers, antioxidants, tonicity agents, bulking agents, lubricating agents, emulsifiers, suspending or viscosity agents, antibacterial agents, chelating agents, preservatives, sweeteners, perfuming agents, flavoring agents, administration aids, and combinations thereof. Some of the excipients or additives may have more than one possible function or use, depending on their properties and the nature of the formulation.

Suitable routes of administration may include, but is not limited to, oral, rectal, transmucosal or intestinal administration or intramuscular, subcutaneous, intramedullary, intrathecal, direct intraventricular, intravenous, intravitreal, intraperitoneal, intranasal, or intraocular injections.

The pharmaceutical composition may be in the forms normally employed, such as tablets, capsules, powders, syrups, solutions, suspensions and the like, may contain flavorants, sweeteners etc. in suitable solid or liquid carriers or diluents, or in suitable sterile media to form injectable solutions or suspensions. Pharmaceutical compositions of the present invention may be manufactured by processes well known in the art, e.g., by means of conventional mixing, dissolving, granulating, drageemaking, levigating, emulsifying, encapsulating, entrapping or lyophilizing processes.

Pharmaceutical compositions for use in accordance with the present invention may be formulated in any conventional manner using one or more physiologically acceptable carriers comprising excipients, diluents and auxiliaries which facilitate processing of the active compounds into preparations which can be used pharmaceutically. The active compound will be present in such pharmaceutical compositions in the amounts sufficient to provide a desired dosage. Proper formulation is dependent upon the route of administration chosen.

Suitable routes of administration include systemic, such as orally or by parenteral administration such as subcutaneous, intramuscular, intravenous and intradermal routes. For example, for oral administration, the pharmaceutical composition may be formulated by combining the active compounds, i.e. the disclosed inhibitor alone or in combination with any of the disclosed compounds, with pharmaceutically acceptable carriers well known in the art. Such carriers enable the compounds of the invention to be formulated as tablets, pills, lozenges, dragees, capsules, liquids, gels, syrups, slurries, suspensions and the like, for oral ingestion by a patient. Pharmaceutical preparations for oral use can be made using a solid excipient, optionally grinding the resulting mixture, and processing the mixture of granules, after adding other suitable auxiliaries if desired, to obtain tablets or dragee cores. Useful excipients are as outlined above and, in particular, fillers such as sugars, including lactose, sucrose, mannitol, or sorbitol, cellulose preparations such as, for example, maize starch, wheat starch, rice starch and potato starch and other materials such as gelatin, gum tragacanth, methyl cellulose, hydroxypropylmethyl-cellulose, sodium carboxymethylcellulose, and/or polyvinyl-pyrrolidone (PVP). If desired, disintegrating agents may be added, such as cross-linked polyvinyl pyrrolidone, agar, or alginic acid. For parenteral administration, the active compounds can be combined with sterile aqueous or organic media to form injectable solutions or suspensions. For example, solutions in sesame or peanut oil, aqueous propylene glycol and the like can be used. The injectable solutions prepared in this manner can then be, administered intravenously, intraperitoneally, subcutaneously, or intramuscularly.

While the inhibitor of PERK gene may be any one of the inhibitors disclosed above, the present invention also contemplates other inhibitors of PERK gene. To aid in the discovery of other inhibitors of PERK gene, there is provided, in one embodiment, a method of identifying a compound that modulates expression of PERK gene in a cell, the method comprising:
a. exposing cells expressing the PERK gene with a test compound;
b. determining the expression level of the PERK gene in the cells which were exposed to the test compound under (a);
c. comparing the level of expression of the PERK gene determined under (b) with the expression of the PERK gene in control cells which were not exposed to the test compound; wherein a difference in the expression level between the cells under (b) compared to the control cells identifies the compound that modulates expression of the PERK gene in a cell.

The difference in the expression level may be a positive difference. That is, the expression level of the PERK gene of the control cells is higher than the expression level of the PERK gene determined under step (b) above, indicating that the test compound is an inhibitor of the PERK gene and down-regulates the expression of the PERK gene.

Conversely, the difference in the expression level may be a negative difference. That is, the expression level of the PERK gene of the control cells is lower than the expression level of the PERK gene determined under step (b) above, indicating that the test compound does not exhibit inhibition of the PERK gene and therefore does not down-regulate the expression of the PERK gene.

The disclosed method of identifying a compound that modulates expression of PERK gene in a cell may permit the screening of potential inhibitors of the PERK gene.

While the inhibitor of PERK protein may be any one of the inhibitors disclosed above, the present invention also contemplates other inhibitors of PERK protein. To aid in the discovery of other inhibitors of PERK protein, there is provided, in one embodiment, a method of identifying a compound that modulates the amount or activity of PERK protein comprised in a cell, the method comprising:
a. exposing cells expressing PERK protein with a test compound;
b. determining the amount or activity of PERK protein in the cells which were exposed to the test compound under (a);
c. comparing the amount or activity of PERK protein determined under (b) with the activity of PERK protein in control cells not exposed to the test compound; wherein a difference in the amount or activity of PERK protein between the cells under (b) compared to the control cells identifies the compound that modulates the amount of PERK protein in the cells.

In another embodiment, there is also provided a method of identifying a compound that modulates the amount of PERK protein comprised in a cell, the method comprising:
a. exposing cells expressing PERK protein with a test compound;
b. determining the amount of PERK protein in the cells which were exposed to the test compound under (a);
c. comparing the amount of PERK protein determined under (b) with the amount of PERK in control cells not exposed to the test compound; wherein a difference in the amount of PERK protein between the cells under (b) compared to the control cells identifies the compound that modulates the amount of PERK protein in the cells.

The difference in the expression level may be a positive difference. That is, the expression level of the PERK protein of the control cells is higher than the expression level of the PERK protein determined under step (b) above, indicating that the test compound is an inhibitor of PERK protein and down-regulates the amount or activity of the PERK protein.

Conversely, the difference in the expression level may be a negative difference. That is, the expression level of the PERK protein of the control cells is lower than the expression level of the PERK protein determined under step (b) above, indicating that the test compound does not exhibit inhibition of the PERK protein and therefore does not down-regulate the amount or activity of the PERK protein.

The disclosed method of identifying a compound that modulates the amount or activity of PERK protein in a cell may permit the screening of potential inhibitors of the PERK protein.

The activity of PERK protein may be determined by using any one of the following methods:
a. in vitro kinase assays, wherein the in vitro kinase assay is optionally using a known substrate such as the identified PERK phosphorylation sites on PERK (T980) on eIF2a (S51), and/or the novel site(s) identified in Drosophila FOXO (as shown in Table 1 below) or in human Foxo1 (S303);
b. a cell based assay for FOXO activity, wherein the cell based assay optionally includes luciferase assays using a FOXO reporter transgene in human or animal cells;
c. a cell based assay for nuclear localization of FOXO, such as a cell based assay referred to in Example 1 below;
d. a cell based assay based on expression of known FOXO target genes, such as a cell based assay referred to in Example 2 or Examples 7A and 7B below;
e. a cell based or in vitro assay based on PERK protein activity optionally measured by a phospho-specific antibody to the auto-phosphorylation site on PERK (T980), to the PERK site S51 on eIF2a, or to S303 on human Foxo1 (or optionally any of the other novel PERK sites identified or predicted on any of the human FOXO proteins).

In vitro kinase assays and cell based assays provide a high throughput method for screening for inhibitors of PERK protein and are suitable for an initial screen of candidate inhibitors. An in vitro kinase assay is a lab-based technique to study the activity of a kinase of interest bound to an antibody with a target substrate. A cell based assay is commonly used to refer to any assay based on some measurement of a living cell.

Known FOXO target genes are, for example, 4E-BP (a regulator of overall translation levels in cells), CCAAT/enhancer binding protein epsilon (CHOP), Bim (anti- or pro-apoptotic regulators) and Growth Arrest and DNA Damage gene (GADD45).

While there is provided inhibitors of PERK gene and inhibitors of PERK protein that are useful in the treatment or prevention of insulin resistance syndrome, the disclosed inhibitors may not be effective in certain individuals. Accordingly, to aid in predicting whether a subject is receptive to the disclosed treatment, there is provided, in one embodiment, a prognostic method for determining the receptiveness of a patient suffering from insulin resistance syndrome for a treatment with a PERK inhibitor, wherein the method comprises identifying and determining the PERK activity in the patient, wherein an increased PERK activity indicates that the person may be receptive for a treatment with a PERK inhibitor.

Subjects that may be receptive may also benefit from inhibitors of ER stress, which include PERK inhibitors.

The term “prognosis”, or grammatical variants thereof, as used herein refers to a prediction of the probable course and outcome of a clinical condition or disease. A prognosis of a patient is usually made by evaluating factors or symptoms of a disease that are indicative of a favorable or unfavorable course or outcome of the disease. The term “prognosis” does not refer to the ability to predict the course or outcome of a condition with 100% accuracy. Instead, the term “prognosis” refers to an increased probability that a certain course or outcome will occur; that is, that a course or outcome is more likely to occur in a patient exhibiting a given condition, when compared to those individuals not exhibiting the condition.

By the term “suffering from insulin resistance syndrome” is meant that the patient has already been diagnosed, or is suspected to be suffering from insulin resistance syndrome.

The increased PERK protein activity may be determined by a phospho-specific antibody to the auto-phosphorylation site on PERK protein (T980), or to the PERK phosphorylation site S51 on eIF2α, or to the PERK phosphorylation site S303 on human Foxo1 (or optionally any of the other novel PERK sites identified or predicted on any of the human FOXO proteins).

The present inventors have now found that PERK-dependent phosphorylation of FOXO drives FOXO into the nucleus, thereby leading to the conclusion that the ratio of AKT-site phosphorylation to PERK-site phosphorylation is informative.

Accordingly, in one embodiment, there is provided a prognostic method for determining the receptiveness of a patient suffering from insulin resistance syndrome for a treatment with a PERK inhibitor, wherein the method comprises measuring activity of a protein kinase AKT and/or PI3Kinase activity in a subject, wherein in comparison to a control a lowered AKT activity and/or lowered PI3Kinase activity indicates that the patient may be receptive for a treatment with a PERK inhibitor.

The control may be the AKT or PI3K activity in a non-diabetic individual.

In one embodiment, there is provided a prognostic method for determining the receptiveness of a patient suffering from insulin resistance syndrome for a treatment with a PERK inhibitor, wherein the method comprises measuring the relative levels of phosphorylation on the AKT site and on the PERK site(s), wherein a lower ratio of AKT site phosphorylation to PERK site phosphorylation indicates that the patient is receptive for a treatment with a PERK inhibitor.

The determination whether the ratio of AKT site phosphorylation to PERK site phosphorylation is lowered may be carried out by comparing the measured ratio with the ratio determined in a healthy non-diabetic population.

In one embodiment, there is provided a kit for use in treating or preventing insulin resistance syndrome in a patient, said kit comprises one of the following selected from the group consisting of a siRNA as disclosed herein, an antibody as disclosed herein, an organic molecule as disclosed herein and a pharmaceutical composition as disclosed herein.

In one embodiment, there is provided a kit for determining whether a patient suffering from insulin resistance syndrome is receptive for a treatment with a PERK inhibitor, wherein the kit comprises:
a. antibodies specific to one or more of the AKT phosphorylation site(s) on one or more of the human FOXO proteins; and
b. antibodies specific for one or more of the PERK phosphorylation site(s) on one or more of the human FOXO proteins or for one or more of the PERK phosphorylation site(s) on PERK protein or on eIF2a.

The antibodies may be used to determine the ratio of AKT/PERK phosphorylation on FOXO proteins. Assays include, but are not limited to, immunofluorescence and immunoblotting, or enzyme-linked immunosorbent assay (ELISA), or other immobilized antibody method based on antigen capture on samples of patient material. Other methods such as mass spectrometry are possible, but may not be practically useful in a clinical setting. The ratio of AKT/PERK phosphorylation on FOXO proteins may be compared to normal non-diabetic controls. A ratio lower than the controls would indicate that the patient may be a good candidate for treatment with a PERK inhibitor.

The invention illustratively described herein may suitably be practiced in the absence of any element or elements, limitation or limitations, not specifically disclosed herein. Thus, for example, the terms “comprising”, “including”, “containing”, etc. shall be read expansively and without limitation. Additionally, the terms and expressions employed herein have been used as terms of description and not of limitation, and there is no intention in the use of such terms and expressions of excluding any equivalents of the features shown and described or portions thereof, but it is recognized that various modifications are possible within the scope of the invention claimed. Thus, it should be understood that although the present invention has been specifically disclosed by preferred embodiments and optional features, modification and variation of the inventions embodied therein herein disclosed may be resorted to by those skilled in the art, and that such modifications and variations are considered to be within the scope of this invention.

The invention has been described broadly and generically herein. Each of the narrower species and subgeneric groupings falling within the generic disclosure also form part of the invention. This includes the generic description of the invention with a proviso or negative limitation removing any subject matter from the genus, regardless of whether or not the excised material is specifically recited herein.

Other embodiments are within the following claims and non-limiting examples. In addition, where features or aspects of the invention are described in terms of Markush groups, those skilled in the art will recognize that the invention is also thereby described in terms of any individual member or subgroup of members of the Markush group.

### EXPERIMENTAL SECTION

Non-limiting examples of the invention and comparative examples will be further described in greater detail by reference to specific Examples, which should not be construed as in any way limiting the scope of the invention.

The following Examples were carried out based on the fact that the insulin signaling pathway is conserved throughout the animal kingdom. It is therefore possible to perform genetic screens in Drosophila to identify new regulatory mechanisms. Accordingly, the following Examples demonstrate the insulin signaling mechanisms in Drosophila, thereby identifying corresponding mechanisms in mammals.

Drosophila has one FOXO gene. Through a genetic screen in Drosophila, the endoplasmic reticulum stress pathway (ER stress) was identified as an inducer of FOXO activity. On the other hand, ER stress has three effector pathways in mammals: activating transcription factor 6 (ATF6), IRE1 and PERK (also known as eIF2α K₃). ATF6 is an ER stress-regulated transmembrane transcription factor that activates the transcription of ER molecules. IRE1 is a ser/thr protein kinase that possesses endonuclease activity and is important in altering gene expression as a response to ER stress signals.

### Example 1

This example demonstrates that ER stress induces nuclear localization of FOXO in a PERK-dependent manner. A cell-based assay was used to show that ER stress counteracted the effects of insulin signaling, thereby increasing nuclear localization of Drosophila FOXO.

A FOXO-green fluorescent (GFP) fusion protein was expressed in Drosophila S2 cells by transient transfection. The Drosophila cells were grown in serum free medium and deprived of insulin. The transfected cells were treated with 10 μg/ml of insulin for 30 min and the subcellular localization of FOXO-GFP was scored by obtaining images of the FOXO-GFP expressing cells. The insulin treated cells were named sample (b). A control sample (a) was obtained by having no insulin treatment to the transfected cells. In sample (c), ER stress was induced by RNA interference (RNAi) to disrupt endoplasmic-reticulum-associated-protein degradation (ERAD) machinery. The disruption of ERAD machinery was done by treatment of dsRNA to deplete HMG-CoA reductase degradation protein 3 (Hrd3), thereby inducing ER stress. Thereafter, the cells were treated with insulin 4 days after the dsRNA treatment. In sample (d), ER stress was induced by dsRNA treatment to deplete Hrd3. PERK protein was also depleted using dsRNA treatment. Thereafter, the cells were treated with insulin 4 days after the dsRNA treatment.

The results are shown in FIG. 2, where N means predominantly nuclear, C means predominantly cytoplasmic and CN means both nuclear and cytoplasmic. In other words, FIG. 2 shows the percentage of cells in which FOXO-GFP was found primarily in the nucleus, primarily in the cytoplasm or found in both. It can be seen in the control sample in FIG. 2(a) that the FOXO-GFP protein was predominantly nuclear, while it is seen in FIG. 2(b) that the location of FOXO-GFP is cytoplasmic in about 70% of cells and both nuclear and cytoplasmic in about 30% of cells. Accordingly, this evidences that insulin treatment shifts FOXO out of the nucleus and into the cytoplasm. From sample (c) in FIG. 2(c), it can be seen that ER stress (Hrd3 depletion) counteracted the effect of insulin in moving FOXO-GFP out of the nucleus because FOXO activity is now both nuclear and cytoplasmic in about 70% of cells, predominantly cytoplasmic in about 25% of cells and predominantly nuclear in about 5% of cells. However from sample (d) in FIG. 2(d), it can be seen that the depletion of PERK protein using PERK RNA interference even during ER stress retains FOXO predominantly in the cytoplasm in about 70% of cells and FOXO in both the nucleus and cytoplasm in about 30% of cells. Comparing N, CN and C categories of 3 independent experiments for Hrd3 depletion (sample (c)) and co-depletion of Hrd3 and PERK (sample (d)) yielded p-values of p<0.05 for sample (c) and p<0.001 for sample (d). PERK depletion had no effect alone (results not shown).

Accordingly, it is evidenced that depletion of PERK blocked the effect of ER stress on FOXO nuclear localization. It also can be concluded from the results that PERK activity is required to mediate the effects of ER stress to increase nuclear FOXO activity and suggests that PERK activity promotes FOXO activity by promoting nuclear localization of FOXO. On the other hand, the depletion of IRE1 had no effect on the in a comparable experiment (data not shown), showing that in response to ER stress, depletion of IRE1 does not affect the IRE1 effector pathway.

### Example 2

This example demonstrates that PERK potentiates FOXO activity in vivo in Drosophila.

### Example 2A

Quantitative reverse transcription polymerase chain reaction (RT-PCR) was used to measure expression of a known FOXO target, 4E-BP (a regulator of overall translation levels in cells), in RNA samples derived from a Drosophila tissue expressing the nub-Gal4 transgene. This control sample is denoted as “Nub”. Overexpression of an upstream activation sequence (UAS)-PERK transgene in transgenic flies was done by crossing with flies expressing a nub-Gal4 transgene and this sample is denoted as “Nub>PERK”. The RNA was extracted from wing discs of wandering 3^{rd} instar larvae, and treated with DNAse to eliminate genomic DNA contamination. Oligo-dT primers were used for reverse transcription. Results were normalized to Kinesin mRNA levels and to the level of the test RNAs in the nub-Gal4 control samples.

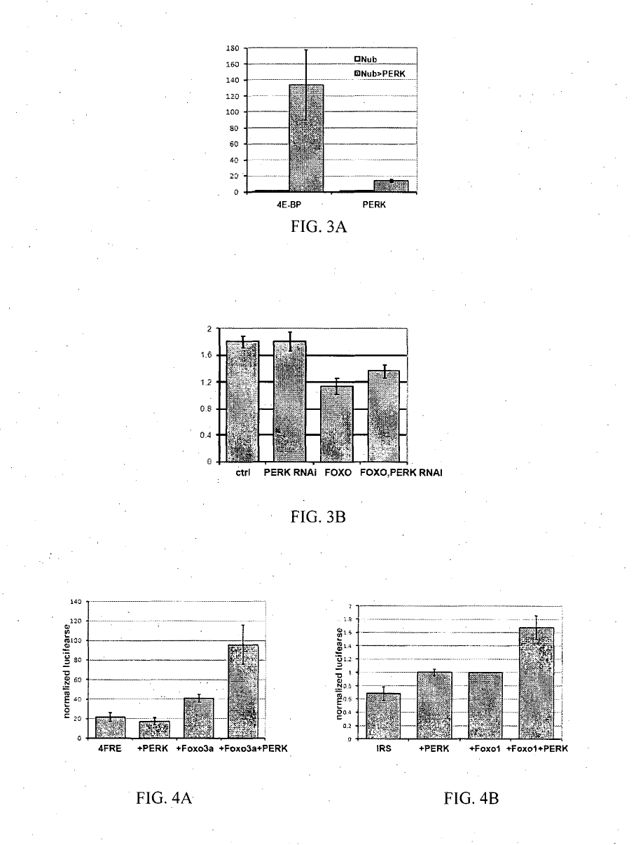

The results are shown in FIG. 3A as fold change relative to the “Nub” control. As seen in FIG. 3A, the levels of 4E-BP are much higher in “Nub>PERK” than in “Nub”. The levels of PERK mRNA shown in FIG. 3A are relative levels normalized to a control mRNA and show the magnitude of increase in PERK mRNA levels resulting from overexpression of the UAS-PERK gene in “Nub>PERK”. It can be seen that the levels of PERK mRNA are also much higher in “Nub>PERK” than in “Nub”. Accordingly, the experimental conditions in this Example positively demonstrate that the overexpression of UAS-PERK in “Nub>PERK” flies increased 4E-BP levels of about 130 fold above the baseline as compared to the “Nub” control flies. Hence, it is evidenced that overexpression of PERK protein in vivo in Drosophila results in an increase in expression of the FOXO target, 4E-BP, and enhances the effects of FOXO overexpression. It thus can be concluded that PERK promotes FOXO activity.

### Example 2B

In this example, relative eye size was used as a measure of the effects of FOXO activity in the control of tissue growth.

The control sample were flies expressing a GMR-Gal4 transgene and is denoted as “ctrl”. Depletion of PERK was accomplished by RNA interference (RNAi) and this sample is denoted as “PERK RNAi”. Overexpression of an upstream activation sequence (UAS)—FOXO transgene in transgenic flies was done by crossing with the control flies and this sample is denoted as “FOXO”. Depletion of PERK was accomplished by coexpression of a UAS RNAi transgene, which expresses a double strand RNA (dsRNA) sequence to target PERK for depletion under Gal4 control, together with UAS-FOXO and GMR-Gal4. This sample is denoted as “FOXO,PERK RNAi”. Digital images of the eye sizes of each sample were taken using a microscope under a standardized magnification. The total area of affected eyes of each genotype sample was measured in pixels using ImageJ, a Java-based image processing program.

The results are shown in FIG. 3B which plots the average eye area in arbitrary units including standard deviation for the samples. As seen in FIG. 3B, when “FOXO” was compared with “FOXO,PERK RNAi”, it is evidenced that depletion of PERK counteracted the effects of FOXO overexpression, as evidenced by an increase in relative eye size. However, when “ctrl” was compared with “PERK RNAi”, there was no effect on relative eye size. Accordingly, it can be concluded that depletion of PERK activity in vivo in Drosophila counteracts the effects of excess FOXO activity. Specifically, depletion of PERK offsets the effects of FOXO overexpression, resulting in an increase in eye area (p<0.05, using Student's T-Test).

From Examples 2A and 2B, it can be concluded that PERK promotes FOXO activity. Further, from both Examples 1 and 2, it can likely be concluded that the promotion of FOXO activity is done by an increase in the movement of FOXO into the nucleus where it can act as a transcription factor.

### Example 3

This example demonstrates that human PERK protein promotes human Foxo1 and Foxo3a activity.

FOXO proteins are transcription factors. To measure the effects of human PERK on human FOXO activity, FOXO luciferase reporters transfected into Michigan Cancer Foundation-7 (MCF-7) cells (a breast cancer cell line) were used. The FOXO luciferase reporters determine FOXO gene expression as measured by luciferase levels.

### Example 3A

In Example 3A, control cells were transfected to express a human Foxo3a firefly luciferase reporter alone, denoted as “4FRE”. The control cells were further cotransfected to express Foxo3a (denoted as “+Foxo3a”), PERK (denoted as “+PERK”), or both Foxo3a and PERK together (denoted as “+Foxo3a+PERK”). A renilla luciferase reporter was co-transfected to assess transfection efficiency. Cells were grown in Dulbecco's modified Eagle's Medium (DMEM) supplemented with 10% fetal bovine serum (FBS). Cell transfection was done using FuGENE® 6 reagents (Roche Applied Science, Germany). Cells were lysed and luciferase assays performed 3 days after transfection using Promega Dual-Luciferase® assay kit (Promega Corporation, Wisconsin, United States of America) in accordance with the manufacturer's instructions. Normalized luciferase levels of the samples were measured and the results are shown in FIG. 4A. As seen in FIG. 4A, expression of Foxo3a increased reporter gene expression as measured by an increase in luciferase levels in the sample “+Foxo3a” as compared to “4FRE” control. PERK expression alone had little effect in the sample “+PERK”. However, co-expression of PERK potentiated the effects of Foxo3a in the sample “+Foxo3a+PERK”.

### Example 3B

In Example 3B, control cells were transfected to express a human Foxo1 luciferase reporter alone, denoted as “IRS”. The control cells were further cotransfected to express Foxo1 (denoted as “+Foxo1”), PERK (denoted as “+PERK”), or both Foxo1 and PERK together (denoted as “+Foxo1+PERK”). A renilla luciferase reporter was co-transfected to assess transfection efficiency. Cells were grown in DMEM media supplemented with 10% FBS. Cell transfection was done using FuGENE® 6 reagents. Cells were lysed and luciferase assays performed 3 days after transfection. Normalized luciferase levels of the samples were measured and the results are shown in FIG. 4B. As seen in FIG. 4B, PERK expression increased reporter activity, presumably acting on endogenous Foxo1. Addition of Foxo1 increased reporter activity in the sample “+Foxo1” as compared to “IRS” control. Co-expression of PERK with Foxo1 increased reporter activity and potentiated the effect of Foxo1 on the Foxo1 luciferase reporter in the sample “+Foxo1+PERK”.

From Examples 3A and 3B, it can be concluded that increased PERK activity potentiated the effects of human FOXO.

### Example 4

This example demonstrates that depletion of PERK reduces FOXO activity in human AGS cells.

The expression of certain mRNAs was measured by quantitative reverse transcription polymerase chain reaction (RT-PCR). In this example, the mRNA was extracted and purified using RNeasy Mini Kit (Qiagen, Netherlands) with DNase to eliminate genomic DNA contamination. Reverse transcription to synthesize the first strand utilized oligo-dT primers. The mRNAs measured are known FOXO targets: CCAAT/enhancer binding protein epsilon (CHOP), Bim (anti- or pro-apoptotic regulators), Growth Arrest and DNA Damage gene (GADD45) and PERK. Bip2, which is not a FOXO target, was also measured as a control. Results were normalized to Kinesin mRNA.

### Example 4A

Dimethyl sulfoxide (DMSO)-treated human AGS cells were used as a control, denoted as “ctrl”. The human AGS cells were grown in Roswell Park Memorial Institute (RPMI) media supplemented with 10% FBS, and treated with 10 μg/ml tunicamycin dissolved in DMSO at 1 mg/ml for 4 hours to induce ER stress. The tunicamycin treated cells are denoted as “TM”.

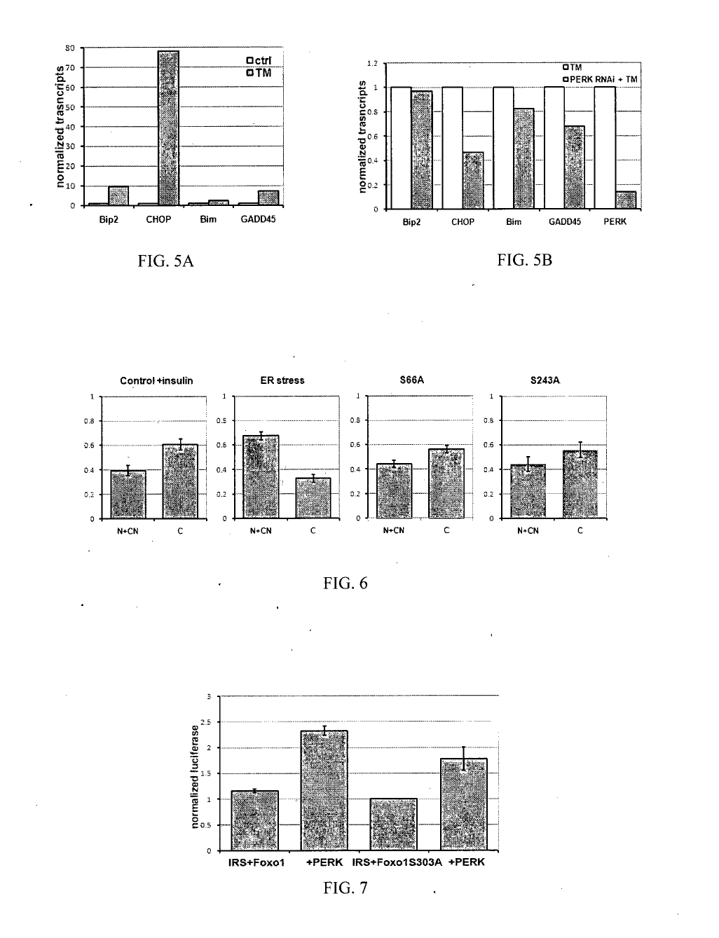

Quantitative RT-PCR was performed to show the relative level of the indicated human mRNA targets. The results are shown in FIG. 5A where normalized levels of mRNA transcripts were measured for the “TM” cells compared to the “ctrl” cells. It can be seen from FIG. 5A that the “TM” cells had significantly higher levels of Bip2, CHOP, Bim and GADD45 as compared to the “ctrl” cells. It can be concluded that ER stress induces the expression of Bip2, CHOP, Bim and GADD45.

### Example 4B

The tunicamycin treated (“TM”) cells were further treated with siRNA treatment to deplete PERK from human AGS cells. The siRNA sequence used was 5′-CAAACUGUAUAACGGUUUATT-3′ (SEQ ID NO: 1). The siRNA was transfected into cells using HiPerFect reagent (Qiagen, Netherlands) for 3 days before tunicamycin treatment. The PERK-depleted cells are denoted as “PERK RNAi+TM”.

Quantitative RT-PCR was performed to show the relative level of the indicated human mRNA targets. The results are shown in FIG. 5B where normalized levels of the target mRNA transcripts were measured for the “TM” cells compared with the “PERK RNAi+TM” cells. The levels of these target mRNAs in PERK-depleted (“PERK RNAi+TM”) cells were normalized to the levels of tunicamycin-treated (“TM”) control cells.

It can be seen from FIG. 5B that the PERK levels decreased more than 80% in the “PERK RNAi+TM” cells as compared to the “TM” cells. As previously concluded in Example 1, ER stress induces nuclear FOXO activity and that PERK promotes FOXO activity. Hence, in this example, PERK depletion down-regulates FOXO activity, thereby reducing the degree of induction of Foxo1 targets CHOP, Bim and GADD45 when comparing “PERK RNAi+TM” cells with “TM” cells. However, the levels of Bip2 transcripts were comparable between both sample cells. This is because Bip2 is not a FOXO target and therefore was not affected by the depletion of PERK.

Examples 4A and 4B thus demonstrate that reduction of PERK activity can reduce FOXO activity in humans. Accordingly, inhibition of PERK provides a novel mechanism to control FOXO activity.

### Example 5

In this example, the hypothesis that PERK phosphorylates FOXO to promote nuclear entry was tested.

A Drosophila FOXO-GFP fusion protein was expressed in Drosophila cells. The cells were treated with 10 μg/ml tunicamycin for 4 hours to induce ER stress, which in turn induces PERK activity. The control cells were not treated with tunicamycin. The purified FOXO-GFP protein was then examined by mass spectrometry to identify sites of phosphorylation that were induced by ER stress.

Table 1 below lists the results of this example. Drosophila FOXO amino acid residues showing increased phosphorylation in response to ER stress was identified. Specifically, the first phosphorylation site is at the amino acid serine at position 66 (S66), the second phosphorylation site is at the amino acid threonine at position 222 (T222), the third phosphorylation site is at the amino acid serine at positions 226 and 227 (S226/S227), the fourth phosphorylation site is at the amino acid serine at position 243 (S243) and the fifth phosphorylation site is at the amino acid serine at position 263 (S263).

TABLE 1

| Site number | Drosophila |
|---|---|
| 1 | Serine 66 |
| 2 | Threonine 222 |
| 3 | Serine 226 |
| 3 | Serine 227 |
| 4 | Serine 243 |
| 5 | Serine 263 |

### Example 6

This example tests the functions of the identified phosphorylation sites in Drosophila.

The identified Serine or Threonine residues were individually mutated to Alanine in Drosophila FOXO and the mutant proteins were tested for ER stress induced nuclear localization. The phosphorylation sites 2, 3 and 5 mutants, labeled as “T222A”, “S263A” and “S226A/S227A”, behaved like the native FOXO, i.e. as T222, S263 and S226/S227 respectively. S66A (site 1) and S243A (site 4) mutant proteins were refractory to ER stress induced nuclear localization, i.e. there was no induced nuclear localization. This indicates that the presence of a phosphorylatable Serine residue at positions 66 and 243 is required for ER stress induced movement of Drosophila FOXO into the nucleus. The other sites do not appear to be required.

The assay described in Example 1 was repeated here, except that the predominantly nuclear (N) and both nuclear and cytoplasmic (NC) data were combined in this example. Example 1 was also repeated to test the mutant proteins expressed in Drosophila cells.

The results are shown in FIG. 6. This example reinforced that insulin treatment (sample denoted as “Control+insulin”) promoted cytoplasmic localization of FOXO-GFP, while ER stress (sample denoted as “ER stress”) counteracted the effects of insulin to promote nuclear localization. The FOXO-GFP mutant proteins “S66A” and “S243A” were treated with RNAi to disrupt ERAD machinery and to induce ER stress but were refractory to the effects of ER stress. Accordingly, ER stress did not substantially counteract the effects on insulin in “S66A” and “S243A” and thus, there was little movement from the cytoplasm into the nucleus, as compared to the “ER stress” sample. It can thus be concluded that Drosophila FOXO phosphorylation sites 1 and 4, i.e. S66 and 5243, are required to induce movement of Drosophila FOXO into the nucleus.

### Example 7

In this example, the mutant phosphorylation site in human Foxo1 was validated.

Residue S303 in human Foxo1 was mutated to Alanine (corresponding to phosphorylation site 4, S243, in Drosophila FOXO). The assay described in Example 3B was repeated here for samples “IRS+Foxo1” and “+PERK” and was repeated for the mutant human Foxo1 (denoted as “IRS+Foxo1 S303A” and “+PERK”).

Activity of human Foxo1 S303A was compared to Foxo1 using the luciferase reporter assay. The results are shown in FIG. 7. As shown in FIG. 7, “IRS+Foxo1S303A” was slightly less effective than the native Foxo1 protein “IRS+Foxo1” (p<0.05) when expressed on its own. When Foxo1 is co-expressed with PERK, the activity of human Foxo1 was enhanced by about 2 fold, when comparing “IRS+Foxo1” with “+PERK”. However, the effect of PERK was attenuated when co-expressed with Foxo1 S303A compared to the native Foxo1 protein (P<0.05), evidenced by the lower levels of normalized luciferase in the “+PERK” samples.

This indicates that S303 in human Foxo1 plays an important role in mediating the effects of PERK on Foxo1 activity. The fact that PERK can still induce activity of Foxo1 S303A suggests that there may be additional sites for PERK-dependent phosphorylation.

### Example 8

H1299 cells (a human non-small cell lung carcinoma cell line derived from the lymph node) were transfected to express V5-epitope tagged Foxo1 or Foxo3 as Foxo1-V5 or Foxo3-V5 for 2 days and subjected to ER stress by treatment with 10 μg/ml of tunicamycin for 4 hours in DMEM.

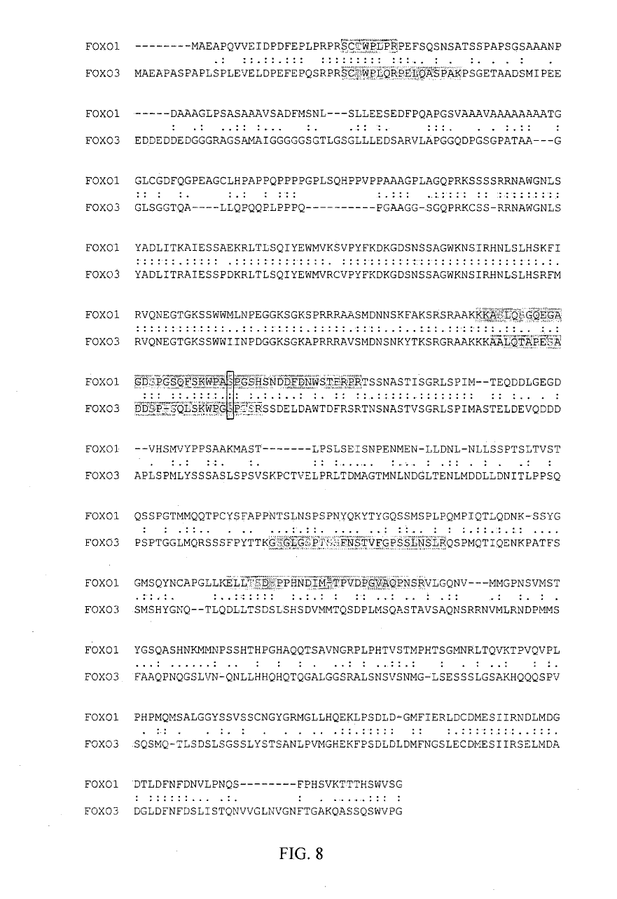

The V5 tagged proteins were immunopurified and examined for phosphorylation. Mass spectrometric analysis identified 8 phosphorylated sites in Foxo1 and 12 sites in Foxo3 in the ER-stress induced H1299 cells. The DNA sequences of Foxo1 and Foxo3 are shown in FIG. 8 with phosphorylated sites shown in red and the corresponding phospho-peptides are shaded in grey. The full DNA sequences of the human Foxo1 and Foxo3 gene are represented as SEQ ID NO: 2 and SEQ ID NO: 3 respectively in the sequence listing.

The cDNA of the human Foxo1 gene has an accession number of CCDS9371.1. The gene encoding the human Foxo1 protein has an accession number of Q12778. The cDNA of the human Foxo3 gene has an accession number of CCDS5068.1. The gene encoding the human Foxo3 protein has an accession number of 043524.

### Example 9

The phosphorylated residues S298 in Foxo1 and S294/T296/S297 in Foxo3 map to a region of the proteins corresponding to the region of S243 in Drosophila FOXO.

To test its function, Serine 298 was converted to Alanine in Foxo1 (“S298A”) and tested for responsiveness to PERK. H1299 cells were transfected to express natural Foxo1 (“FOXO1”) or the S298A mutant version of Foxo1 (“S298A”) and each were co-transfected to overexpress PERK (“FOXO1+PERK” and “S298A+PERK”). This Foxo1 luciferase reporter determines Foxo1 protein activity as measured by luciferase levels and the induction of the Foxo1 luciferase reporter was assayed.

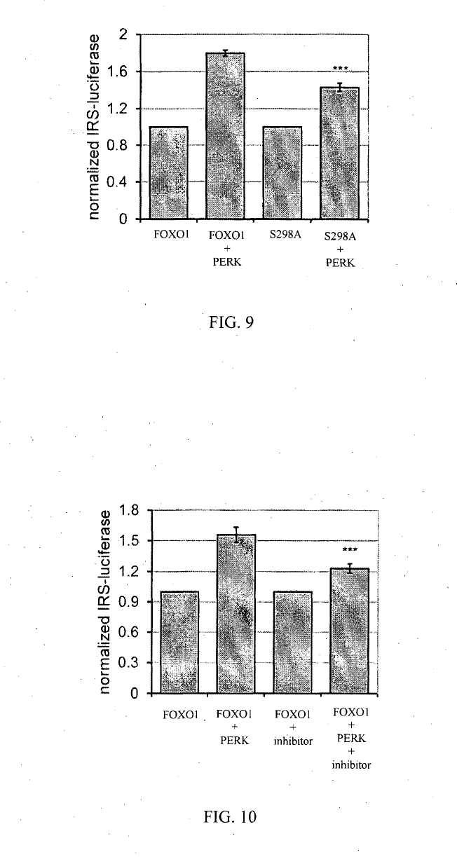

The results are shown in FIG. 9 where normalized luciferase levels were measured for the samples. The levels of luciferase were normalized to the levels of FOXO1 alone. It can be seen from FIG. 9 that the activity of the S298A mutant form of Foxo1 (“S298A”) was comparable to that of the normal intact form of Foxo1 (“FOXO 1”) in the reporter assay without added PERK. However, “S298A” showed a lower response to PERK overexpression as compared to the normal Foxo1, when comparing “S298A+PERK” with “FOXO1+PERK”.

It can be concluded that S298 is one of the sites on human Foxo1 that contributes to mediating the effects of PERK on Foxo1 activity.

### Example 10

Human H1299 cells were transfected to express human Foxo1 (“FOXO1”) and co-transfected to express PERK (“FOXO1+PERK”). The transfected H1299 cells were treated with 1 μM PERK kinase inhibitor (“FOXO1+inhibitor” and “FOXO1+PERK+inhibitor”). The PERK kinase inhibitor has the chemical formula: 5-Bromo-N₄-2-pyridinyl-N₂-[3-(1,2,3,6-tetrahydro-4-pyridinyl)-1H-indol-5-yl]-2,4-pyrimidinediamine. The activity of the Foxo1 luciferase reporter gene was then measured in terms of normalized IRS-luciferase levels.

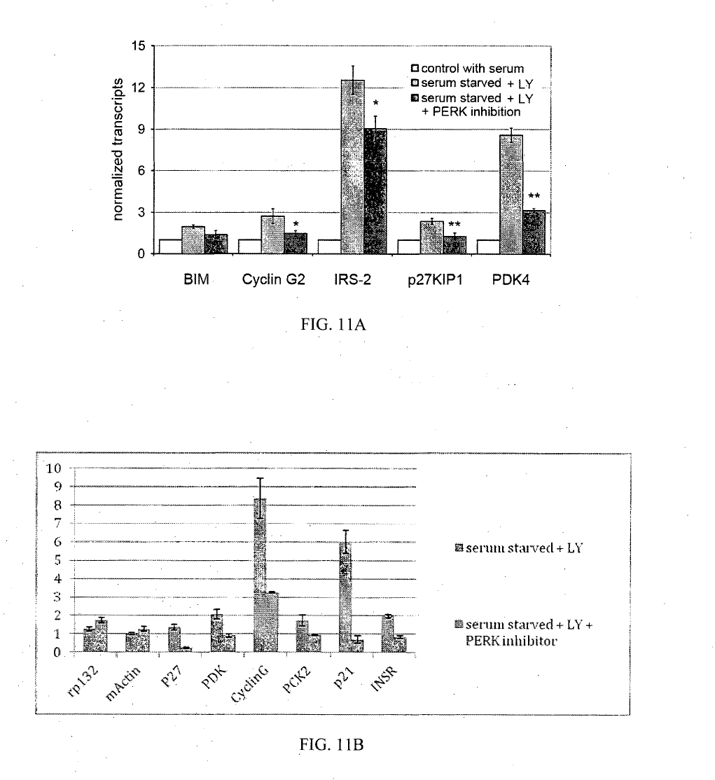

The results are shown in FIG. 10. It can be seen from FIG. 10 that PERK potentiates the effect of Foxo1, when comparing “FOXO1” with “FOXO1+PERK”. The PERK inhibitor did not affect the activity of Foxo1 alone, when comparing “FOXO 1” with “FOXO1+inhibitor”. However, when comparing “FOXO1+PERK” with “FOXO1+PERK+inhibitor”, it can be seen that the partial inhibition of PERK activity reduced the level of Foxo1 activity.

Accordingly, it can be concluded that the PERK kinase inhibitor partially inhibits PERK activity, thereby down-regulating Foxo1 activity.

### Example 11

To test the effects of PERK inhibition on endogenous Foxo1 targets, the cells were serum starved to remove growth factors and treated with the PI3K inhibitor LY294002 (50 μM) to further reduce AKT activity (“serum starved+LY”) for 4 hours. This treatment can be viewed as a model for extreme insulin resistance, in that it removes the capacity of the cell to respond insulin signaling by activating AKT. Control cells were grown in DMEM supplemented with 10% FBS (“control with serum”). The endogenous human FOXO targets are BIM gene (a pro-apoptotic member of the BCL-2 protein family), Cyclin G2 gene (encoding a cyclin that blocks cell cycle entry), insulin receptor substrate 2 gene (IRS-2; encoding a cytoplasmic signaling molecule that mediates effects of insulin), p27KIP1 gene (encoding a cell cycle regulatory protein), pyruvate dehydrogenase lipoamide kinase isozyme 4 gene (PDK4; encoding a protein that inhibits the pyruvate dehydrogenase complex), phosphoenolpyruvate carboxykinase 2 (PCK2; encoding a mitochondrial enzyme), p21 gene (encoding a cyclin-dependent kinase inhibitor) and insulin receptor gene (INSR). The rp132 gene (a ribosomal protein gene) and the mActin gene (encoding a muscle-specific type actin) are not FOXO targets and were used as controls.

It is to be noted that in this example, the cells were not transfected to overexpress PERK, to prove that endogenous PERK activity contributes to FOXO activation under conditions where the inhibitory effects of insulin/AKT signaling are removed.

### Example 11A

The serum starved and PI3K inhibited cells were further treated with PERK kinase inhibitor 5-Bromo-N₄-2-pyridinyl-N₂— [3-(1,2,3,6-tetrahydro-4-pyridinyl)-1H-indol-5-yl]-2,4-pyrimidinediamine, denoted as “serum starved+LY+PERK inhibition”.

The results are shown in FIG. 11A. It can be seen from FIG. 11A that reduction of AKT activity generally increased Foxo1 activity. Further, PERK inhibition generally counteracted the effects of AKT reduction, thereby reducing the target genes expression in Michigan Cancer Foundation-7 (MCF-7) cells (a breast cancer cell line).

### Example 11B

Example 11A was repeated with HEPG2 hepatocyte cells instead.

The results are shown in FIG. 11B where PERK inhibition also counteracted the effects of AKT reduction and reduced the target genes expression. In contrast, PERK inhibition did not reduce the expression of rp132 and mActin as they are not FOXO target genes.

### Example 12

This example demonstrates that the inhibition of PERK overcomes insulin resistance.

Resistance to insulin is a hallmark of Type 2 diabetes, and obesity-related metabolic syndrome is accompanied by an acquired insulin resistance.

### Example 12A

HEPG2 liver cells were treated with 0.75 mM of palmitate, a saturated fatty acid, for 17 hours to induce insulin resistance, and the effects of PERK inhibition were assessed by monitoring Foxo1 target gene expression. The Foxo1 target genes are pyruvate dehydrogenase lipoamide kinase isozyme 4 gene (PDK4; encoding a protein that inhibits the pyruvate dehydrogenase complex) and phosphoenolpyruvate carboxykinase 1 (PCK1; encoding a mitochondrial enzyme).

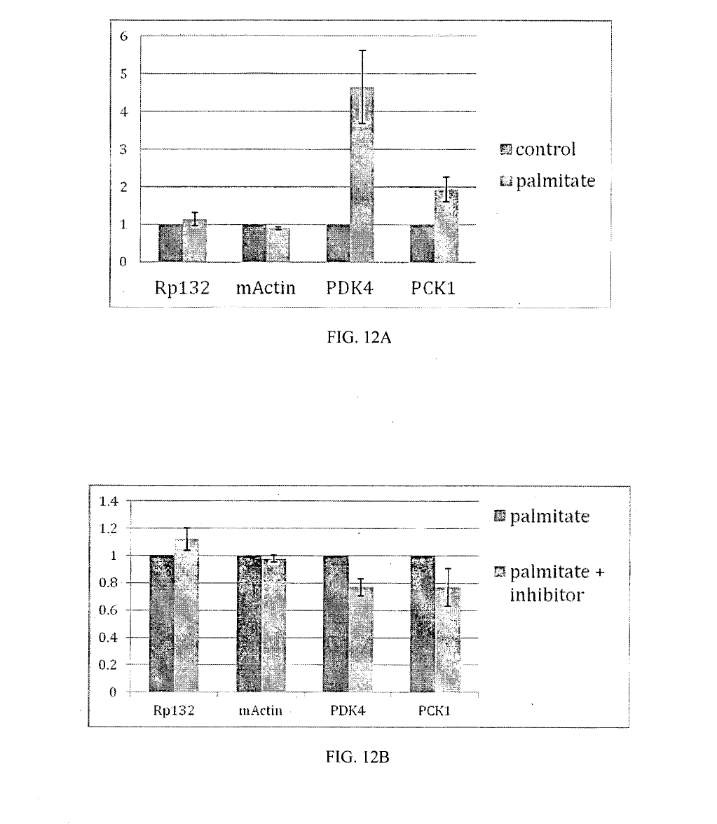

The results are shown in FIG. 12A, where the data was normalized to Kinesin mRNA levels and to the level of the rp132 control mRNAs. It can be seen that palmitate treated cells had substantially higher levels of expression of the Foxo1 targets PDK4 and PCK1 (measured by quantitative real time PCR). However, the levels of rp123 and mActin, which are not Foxo1 targets, were not substantially altered.

It can thus be concluded that saturated fatty acids induces Foxo1 activity as a consequence of induced insulin resistance.

### Example 12B

The palmitate treated cells were further treated by inhibiting PERK kinase inhibitor 5-Bromo-N₄-2-pyridinyl-N₂-[3-(1,2,3,6-tetrahydro-4-pyridinyl)-1H-indol-5-yl]-2,4-pyrimidinediamine.

The results are shown in FIG. 12B, where the data was normalized to Kinesin mRNA levels and to the level of the rp132 control mRNAs. It can be seen that PERK inhibition decreased the expression of the Foxo1 targets, PDK4 and PCK1. However, the levels of rp123 and mActin, which are not Foxo1 targets, were not substantially altered.

It can thus be concluded that a reduction of Foxo1 activity is a consequence of PERK inhibition in cells induced with insulin resistance.

1. A method of treating or preventing insulin resistance syndrome in an animal body by administering an inhibitor of protein kinase RNA-like endoplasmic reticulum kinase (PERK) gene, or a functional variant thereof, or an inhibitor of PERK protein or a functional variant thereof.

2. A method of reducing activity of transcription factors of the FOXO family (Foxo1, 3a, 4 and 6) by administering an inhibitor of protein kinase RNA-like endoplasmic reticulum kinase (PERK) gene, or a functional variant thereof, or an inhibitor of PERK protein or a functional variant thereof.

3. The method of claim 1, wherein the method is used for treating any one of the following conditions which are caused by insulin resistance syndrome: insulin resistance, hypertension, dyslipidemia, Type 2 diabetes or coronary artery disease.

4. (canceled)

5. The method of claim 1, wherein the inhibitor of any of the genes referred to in any one of the preceding claims comprises at least one oligonucleotide or at least one antibody or at least one inorganic molecule or at least one organic molecule.

6. The method of claim 5, wherein the oligonucleotide is an interfering ribonucleic acid, or PNA (protein nucleic acid) or LNA (locked nucleic acid).

7. The method of claim 6, wherein the interfering ribonucleic acid is a small interfering ribonucleic acid (siRNA) or small hairpin ribonucleic acid (shRNA) or micro ribonucleic acid (miRNA).

8. (canceled)

9. The method of claim 7, wherein the siRNA comprises the following sequence: 5′-CAAACUGUAUAACGGUUUATT-3′, or functional variants thereof.

10. (canceled)

11. The method of claim 5, wherein the organic molecule is selected from the group consisting of:
5-bromo-N4-2-pyridinyl-N2-[3-(1,2,3,6-tetrahydro-4-pyridinyl)-1H-indol-5-yl]-2,4-pyrimidinediamine;
1-[5-(4-Amino-7-methyl-7H-pyrrolo[2,3-d]pyrimidin-5-yl)-2,3-dihydro-1H-indol-1-yl]-2-[3-fluoro-5-(trifluoromethyl)phenyl]ethanone;
1-methyl-3-[1-(phenylacetyl)-2,3-dihydro-1H-indol-5-yl]-1H-pyrazolo[3,4-d]pyrimidin-4-amine;
3-{1-[(2,5-difluorophenyl)acetyl]-2,3-dihydro-1H-indol-5-yl}-1-methyl-1H-pyrazolo[3,4-d]pyrimidin-4-amine;
3-[1-(phenylacetyl)-2,3-dihydro-1H-indol-5-yl]-1H-pyrazolo[3,4-d]pyrimidin-4-amine;
7-methyl-5-[1-(phenylacetyl)-2,3-dihydro-1H-indol-5-yl]-7H-pyrrolo[2,3-d]pyrimidin-4-amine;
3-[1-(phenylacetyl)-2,3-dihydro-1H-indol-5-yl]thieno[3,2-c]pyridin-4-amine;
3-{1-[(2,5-difluorophenyl)acetyl]-2,3-dihydro-1H-indol-5-yl}thieno[3,2-c]pyridin-4-amine;
3-[1-(phenylacetyl)-2,3-dihydro-1H-indol-5-yl]-7-(3-pyridinyl)thieno[3,2-c]pyridin-4-amine;
1-methyl-4-{1-[(3-methylphenyl)acetyl]-2,3-dihydro-1H-indol-5-yl}-1H-indazol-3-amine;
3-[1-(phenylacetyl)-2,3-dihydro-1H-indol-5-yl]-7-(4-pyridinyl)thieno[3,2-c]pyridin-4-amine;
3-{1-[(2,5-difluorophenyl)acetyl]-2,3-dihydro-1H-indol-5-yl}-7-(3-pyridinyl)thieno[3,2-c]pyridin-4-amine;
3-{1-[(2,5-difluorophenyl)acetyl]-2,3-dihydro-1H-indol-5-yl}-7-(1H-pyrazol-3-yl)thieno[3,2-c]pyridin-4-amine;
4-{1-[(2,5-difluorophenyl)acetyl]-2,3-dihydro-1H-indol-5-yl}-1-methyl-1H-indazol-3-amine;
3-[1-(phenylacetyl)-2,3-dihydro-1H-indol-5-yl]-7-(1H-pyrazol-4-yl)thieno[3,2-c]pyridin-4-amine;
7-(1-methyl-1H-pyrazol-4-yl)-3-[1-(phenylacetyl)-2,3-dihydro-1H-indol-5-yl]thieno[3,2-c]pyridin-4-amine;
3-{1-[(2-fluorophenyl)acetyl]-2,3-dihydro-1H-indol-5-yl}-1-methyl-1H-pyrazolo[3,4-d]pyrimidin-4-amine;
3-{1-[(3-fluorophenyl)acetyl]-2,3-dihydro-1H-indol-5-yl}-1-methyl-1H-pyrazolo[3,4-d]pyrimidin-4-amine;
1-methyl-3-{1-[(2-methylphenyl)acetyl]-2,3-dihydro-1H-indol-5-yl}-1H-pyrazolo[3,4-d]pyrimidin-4-amine;
1-methyl-3-{1-[(3-methylphenyl)acetyl]-2,3-dihydro-1H-indol-5-yl}-1H-pyrazolo[3,4-d]pyrimidin-4-amine;
3-[1-(phenylacetyl)-2,3-dihydro-4H-indol-5-yl]-7-(1,2,3,6-tetrahydro-4-pyridinyl)thieno[3,2-c]pyridin-4-amine;
3-(1-{[3-(trifluoromethyl)phenyl]acetyl}-2,3-dihydro-1H-indol-5-yl)thieno[3,2-c]pyridin-4-amine;
3-{1-[(2-chlorophenyl)acetyl]-2,3-dihydro-1H-indol-5-yl}thieno[3,2-c]pyridin-4-amine;
3-{1-[(3-chlorophenyl)acetyl]-2,3-dihydro-1H-indol-5-yl}thieno[3,2-c]pyridin-4-amine;
3-(1-{[3-(methyloxy)phenyl]acetyl}-2,3-dihydro-1H-indol-5-yl)thieno[3,2-c]pyridin-4-amine;
3-(1-{[2-(methyloxy)phenyl]acetyl}-2,3-dihydro-1H-indol-5-yl)thieno[3,2-c]pyridin-4-amine;
3-[1-(2-naphthalenylacetyl)-2,3-dihydro-1H-indol-5-yl]thieno[3,2-c]pyridin-4-amine;
3-[1-(phenylacetyl)-2,3-dihydro-1H-indol-5-yl]-7-(4-piperidinyl)thieno[3,2-c]pyridin-4-amine;
7-{3-[(dimethylamino)methyl]phenyl}-3-[1-(phenylacetyl)-2,3-dihydro-1H-indol-5-yl]thieno[3,2-c]pyridin-4-amine;
3-{1-[(2,5-dimethylphenyl)acetyl]-2,3-dihydro-1H-indol-5-yl}-1-methyl-1H-pyrazolo[3,4-d]pyrimidin-4-amine;
3-{1-[(3-fluoro-5-methylphenyl)acetyl]-2,3-dihydro-1H-indol-5-yl}-1-methyl-1H-pyrazolo[3,4-d]pyrimidin-4-amine;
3-{1-[(3,5-dimethylphenyl)acetyl]-2,3-dihydro-1H-indol-5-yl}-1-methyl-4H-pyrazolo[3,4-d]pyrimidin-4-amine;
5-{1-[(2,5-difluorophenyl)acetyl]-2,3-dihydro-1H-indol-5-yl}thieno[2,3-d]pyrimidin-4-amine;
3-{1-[(2,3-difluorophenyl)acetyl]-2,3-dihydro-1H-indol-5-yl}-1-methyl-1H-pyrazolo[3,4-d]pyrimidin-4-amine;
7-methyl-5-{1-[(2-methylphenyl)acetyl]-2,3-dihydro-1H-indol-5-yl}-7H-pyrrolo[2,3-d]pyrimidin-4-amine;
5-{1-[(2-fluorophenyl)acetyl]-2,3-dihydro-1H-indol-5-yl}-7-methyl-7H-pyrrolo[2,3-d]pyrimidin-4-amine;
5-{1-[(3-fluorophenyl)acetyl]-2,3-dihydro-1H-indol-5-yl}-7-methyl-7H-pyrrolo[2,3-d]pyrimidin-4-amine;
3-{1-[(2,3-difluorophenyl)acetyl]-2,3-dihydro-1H-indol-5-yl}thieno[3,2-c]pyridin-4-amine;
7-methyl-5-{1-[(3-methylphenyl)acetyl]-2,3-dihydro-1H-indol-5-yl}-7H-pyrrolo[2,3-d]pyrimidin-4-amine;
3-{1-[(3-fluoro-2-methylphenyl)acetyl]-2,3-dihydro-1H-indol-5-yl}thieno[3,2-c]pyridin-4-amine;
3-{2-[5-(4-aminothieno[3,2-c]pyridin-3-yl)-2,3-dihydro-1H-indol-1-yl]-2-oxoethyl}benzonitrile;
3-{1-[(2-fluoro-5-methylphenyl)acetyl]-2,3-dihydro-1H-indol-5-yl}-1-methyl-1H-pyrazolo[3,4-d]pyrimidin-4-amine;
3-{1-[(2,3-dimethylphenyl)acetyl]-2,3-dihydro-1H-indol-5-yl}-1-methyl-1H-pyrazolo[3,4-d]pyrimidin-4-amine;
3-{1-[(3-chlorophenyl)acetyl]-2,3-dihydro-1H-indol-5-yl}-1-methyl-1H-pyrazolo[3,4-d]pyrimidin-4-amine;
1-methyl-3-(1-{[3-(trifluoromethyl)phenyl]acetyl}-2,3-dihydro-1H-indol-5-yl)-1H-pyrazolo[3,4-d]pyrimidin-4-amine;
7-methyl-5-(1-{[3-(trifluoromethyl)phenyl]acetyl}-2,3-dihydro-1H-indol-5-yl)-7H-pyrrolo[2,3-d]pyrimidin-4-amine;
5-{1-[(3-fluoro-5-methylphenyl)acetyl]-2,3-dihydro-1H-indol-5-yl}-7-methyl-7H-pyrrolo[2,3-d]pyrimidin-4-amine;
5-{1-[(3-chlorophenyl)acetyl]-2,3-dihydro-1H-indol-5-yl}-7-methyl-7H-pyrrolo[2,3-d]pyrimidin-4-amine;
5-{1-[(2-chlorophenyl)acetyl]-2,3-dihydro-1H-indol-5-yl}-7-methyl-7H-pyrrolo[2,3-d]pyrimidin-4-amine;
7-methyl-5-(1-{[2-(methyloxy)phenyl]acetyl}-2,3-dihydro-1H-indol-5-yl)-7H-pyrrolo[2,3-d]pyrimidin-4-amine;
1-methyl-3-(1-{[3-(methyloxy)phenyl]acetyl}-2,3-dihydro-1H-indol-5-yl)-1H-pyrazolo[3,4-d]pyrimidin-4-amine;
7-methyl-5-(1-{[3-(methyloxy)phenyl]acetyl}-2,3-dihydro-1H-indol-5-yl)-7H-pyrrolo[2,3-d]pyrimidin-4-amine;
3-{1-[(2-chlorophenyl)acetyl]-2,3-dihydro-1H-indol-5-yl}-1-methyl-1H-pyrazolo[3,4-d]pyrimidin-4-amine;
1-methyl-3-(1-{[2-(methyloxy)phenyl]acetyl}-2,3-dihydro-1H-indol-5-yl)-1H-pyrazolo[3,4-d]pyrimidin-4-amine;
5-{1-[(3-chloro-5-fluorophenyl)acetyl]-2,3-dihydro-1H-indol-5-yl}-7-methyl-7H-pyrrolo[2,3-d]pyrimidin-4-amine;
3-{1-[(2,5-difluorophenyl)acetyl]-2,3-dihydro-1H-indol-5-yl}furo[3,2-c]pyridin-4-amine;
1-methyl-3-{1-[(2,3,5-trifluorophenyl)acetyl]-2,3-dihydro-1H-indol-5-yl}-1H-pyrazolo[3,4-d]pyrimidin-4-amine;
5-{1-[(2,5-dimethylphenyl)acetyl]-2,3-dihydro-1H-indol-5-yl}-7-methyl-7H-pyrrolo[2,3-d]pyrimidin-4-amine;
3-{1-[(2,5-difluorophenyl)acetyl]-2,3-dihydro-1H-indol-5-yl}-7-(1H-pyrazol-4-yl)furo[3,2-c]pyridin-4-amine;
3-{1-[(3,5-dichlorophenyl)acetyl]-2,3-dihydro-1H-indol-5-yl}-1-methyl-1H-pyrazolo[3,4-d]pyrimidin-4-amine;
5-{1-[(2,5-difluorophenyl)acetyl]-2,3-dihydro-1H-indol-5-yl}-7-methyl-7H-pyrrolo[2,3-d]pyrimidin-4-amine;
3-{1-[(2,5-difluorophenyl)acetyl]-2,3-dihydro-1H-indol-5-yl}-7-(1H-pyrazol-4-yl)thieno[3,2-c]pyridin-4-amine;
3-{1-[(3,5-difluorophenyl)acetyl]-2,3-dihydro-1H-indol-5-yl}-1-methyl-1H-pyrazolo[3,4-d]pyrimidin-4-amine;
5-{1-[(3-methylphenyl)acetyl]-2,3-dihydro-1H-indol-5-yl}-7-(4-piperidinyl)-7H-pyrrolo[2,3-d]pyrimidin-4-amine;
5-{1-[(3-methylphenyl)acetyl]-2,3-dihydro-1H-indol-5-yl}-7-(1-methyl-4-piperidinyl)-7H-pyrrolo[2,3-d]pyrimidin-4-amine;
5-{1-[(3-methylphenyl)acetyl]-2,3-dihydro-1H-indol-5-yl}thieno[2,3-d]pyrimidin-4-amine;
3-{1-[(3-fluoro-5-methylphenyl)acetyl]-2,3-dihydro-1H-indol-5-yl}furo[3,2-c]pyridin-4-amine;
3-{1-[(3-chloro-5-fluorophenyl)acetyl]-2,3-dihydro-1H-indol-5-yl}furo[3,2-c]pyridin-4-amine;
3-{1-[(2-fluoro-5-methylphenyl)acetyl]-2,3-dihydro-1H-indol-5-yl}furo[3,2-c]pyridin-4-amine;
1-methyl-3-{1-[(1-methyl-1H-pyrrol-2-yl)acetyl]-2,3-dihydro-1H-indol-5-yl}-1H-pyrazolo[3,4-d]pyrimidin-4-amine;
3-{1-[(3-chlorophenyl)acetyl]-2,3-dihydro-1H-indol-5-yl}furo[3,2-c]pyridin-4-amine;
5-{1-[(2,3-difluorophenyl)acetyl]-2,3-dihydro-1H-indol-5-yl}-7-methyl-7H-pyrrolo[2,3-d]pyrimidin-4-amine;
5-{1-[(2-fluoro-3-methylphenyl)acetyl]-2,3-dihydro-1H-indol-5-yl}-7-methyl-7H-pyrrolo[2,3-d]pyrimidin-4-amine;
5-{1-[(3-fluoro-2-methylphenyl)acetyl]-2,3-dihydro-1H-indol-5-yl}-7-methyl-7H-pyrrolo[2,3-d]pyrimidin-4-amine;
5-{1-[(2-fluoro-5-methylphenyl)acetyl]-2,3-dihydro-1H-indol-5-yl}-7-methyl-7H-pyrrolo[2,3-d]pyrimidin-4-amine;
3-{1-[(2-fluoro-3-methylphenyl)acetyl]-2,3-dihydro-1H-indol-5-yl}-1-methyl-1H-pyrazolo[3,4-d]pyrimidin-4-amine;
3-{1-[(3-fluoro-2-methylphenyl)acetyl]-2,3-dihydro-1H-indol-5-yl}-1-methyl-1H-pyrazolo[3,4-d]pyrimidin-4-amine;
5-{1-[(2,5-difluorophenyl)acetyl]-2,3-dihydro-1H-indol-5-yl}-7-(1-methyl-4-piperidinyl)-7H-pyrrolo[2,3-d]pyrimidin-4-amine;
5-{1-[(3-chloro-4-fluorophenyl)acetyl]-2,3-dihydro-1H-indol-5-yl}-7-methyl-7H-pyrrolo[2,3-d]pyrimidin-4-amine;
5-{1-[(3-chloro-2-fluorophenyl)acetyl]-2,3-dihydro-1H-indol-5-yl}-7-methyl-7H-pyrrolo[2,3-d]pyrimidin-4-amine;
3-{1-[(3-chloro-4-fluorophenyl)acetyl]-2,3-dihydro-1H-indol-5-yl}-1-methyl-1H-pyrazolo[3,4-d]pyrimidin-4-amine;
3-{1-[(3-chloro-2-fluorophenyl)acetyl]-2,3-dihydro-1H-indol-5-yl}-1-methyl-1H-pyrazolo[3,4-d]pyrimidin-4-amine;
5-{1-[(2,3-dimethylphenyl)acetyl]-2,3-dihydro-1H-indol-5-yl}-7-methyl-7H-pyrrolo[2,3-d]pyrimidin-4-amine;
1-(1-methylethyl)-3-{1-[(3-methylphenyl)acetyl]-2,3-dihydro-1H-indol-5-yl}-1H-pyrazolo[3,4-d]pyrimidin-4-amine;
2-(4-amino-3-{1-[(3-methylphenyl)acetyl]-2,3-dihydro-1H-indol-5-yl}-1H-pyrazolo[3,4-d]pyrimidin-1-yl)ethanol;
5-{1-[(3,5-dimethylphenyl)acetyl]-2,3-dihydro-1H-indol-5-yl}-7-methyl-7H-pyrrolo[2,3-d]pyrimidin-4-amine;
5-{1-[(2,5-difluorophenyl)acetyl]-2,3-dihydro-1H-indol-5-yl}-7-(4-piperidinyl)-7H-pyrrolo[2,3-d]pyrimidin-4-amine;
1-ethyl-3-{1-[(3-methylphenyl)acetyl]-2,3-dihydro-1H-indol-5-yl}-1H-pyrazolo[3,4-d]pyrimidin-4-amine;
3-{1-[(2,5-difluorophenyl)acetyl]-2,3-dihydro-1H-indol-5-yl}-7-methylfluro[3,2-c]pyridin-4-amine;
3-{1-[(2,5-difluorophenyl)acetyl]-2,3-dihydro-1H-indol-5-yl}-1-(1-methylethyl)-1H-pyrazolo[3,4-d]pyrimidin-4-amine;
5-{1-[(3,5-difluorophenyl)acetyl]-2,3-dihydro-1H-indol-5-yl}-7-methyl-7H-pyrrolo[2,3-d]pyrimidin-4-amine;
7-methyl-5-{1-[(2,3,5-trifluorophenyl)acetyl]-2,3-dihydro-1H-indol-5-yl}-7H-pyrrolo[2,3-d]pyrimidin-4-amine;
5-{1-[(3,5-dichlorophenyl)acetyl]-2,3-dihydro-1H-indol-5-yl}-7-methyl-7H-pyrrolo[2,3-d]pyrimidin-4-amine;
7-(3-azetidinyl)-5-{1-[(3-methylphenyl)acetyl]-2,3-dihydro-N-indol-5-yl}-7H-pyrrolo[2,3-d]pyrimidin-4-amine;
5-{1-[(4-fluorophenyl)acetyl]-2,3-dihydro-1H-indol-5-yl}-7-methyl-7H-pyrrolo[2,3-d]pyrimidin-4-amine;
7-methyl-5-{1-[(4-methylphenyl)acetyl]-2,3-dihydro-1H-indol-5-yl}-7H-pyrrolo[2,3-d]pyrimidin-4-amine;
5-{1-[(3-chloro-2,4-difluorophenyl)acetyl]-2,3-dihydro-1H-indol-5-yl}-7-methyl-7H-pyrrolo[2,3-d]pyrimidin-4-amine;
5-(1-{[3-fluoro-5-(trifluoromethyl)phenyl]acetyl}-2,3-dihydro-1H-indol-5-yl)-7-methyl-7H-pyrrolo[2,3-d]pyrimidin-4-amine;
7-[(methyloxy)methyl]-5-{1-[(3-methylphenyl)acetyl]-2,3-dihydro-1H-indol-5-yl}-7H-pyrrolo[2,3-d]pyrimidin-4-amine;
7-methyl-5-{1-[(1-methyl-1H-pyrrol-2-yl)acetyl]-2,3-dihydro-1H-indol-5-yl}-7H-pyrrolo[2,3-d]pyrimidin-4-amine;
5-{1-[(2,5-difluorophenyl)acetyl]-2,3-dihydro-1H-indol-5-yl}-7-(1-methylethyl)-7H-pyrrolo[2,3-d]pyrimidin-4-amine;
5-{1-[(5-chloro-2-fluorophenyl)acetyl]-2,3-dihydro-1H-indol-5-yl}-7-methyl-7H-pyrrolo[2,3-d]pyrimidin-4-amine;
5-{1-[(2,5-difluorophenyl)acetyl]-2,3-dihydro-1H-indol-5-yl}-7-[2-(4-morpholinyl)ethyl]-7H-pyrrolo[2,3-d]pyrimidin-4-amine;
5-{1-[(2,4-difluorophenyl)acetyl]-2,3-dihydro-1H-indol-5-yl}-7-methyl-7H-pyrrolo[2,3-d]pyrimidin-4-amine;
5-{1-[(3,4-difluorophenyl)acetyl]-2,3-dihydro-1H-indol-5-yl}-7-methyl-7H-pyrrolo[2,3-d]pyrimidin-4-amine;
phenylmethyl[2-(4-amino-3-{1-[(2,5-difluorophenyl)acetyl]-2,3-dihydro-1H-indol-5-yl}furo[3,2-c]pyridin-7-yl)ethyl]carbamate;
5-{1-[(2,5-difluorophenyl)acetyl]-2,3-dihydro-1H-indol-5-yl}-7-(3-methylbutyl)-7H-pyrrolo[2,3-d]pyrimidin-4-amine;
5-{1-[(2,5-difluorophenyl)acetyl]-2,3-dihydro-1H-indol-5-yl}-7-[2-(dimethylamino)ethyl]-7H-pyrrolo[2,3-d]pyrimidin-4-amine;
5-{1-[(6-chloro-2-pyridinyl)acetyl]-2,3-dihydro-1H-indol-5-yl}-7-methyl-7H-pyrrolo[2,3-d]pyrimidin-4-amine;
3-{1-[(3-chloro-2,4-difluorophenyl)acetyl]-2,3-dihydro-1H-indol-5-yl}-1-methyl-1H-pyrazolo[3,4-d]pyrimidin-4-amine;
7-(2-aminoethyl)-3-{1-[(2,5-difluorophenyl)acetyl]-2,3-dihydro-1H-indol-5-yl}furo[3,2-d]pyridin-4-amine;
4-amino-3-{1-[(2,5-difluorophenyl)acetyl]-2,3-dihydro-1H-indol-5-yl}furo[3,2-c]pyridine-7-carbonitrile;
5-{1-[(3,5-dimethyl-1H-pyrazol-1-yl)acetyl]-2,3-dihydro-1H-indol-5-yl}-7-methyl-7H-pyrrolo[2,3-d]pyrimidin-4-amine;
5-[4-fluoro-1-(phenylacetyl)-2,3-dihydro-1H-indol-5-yl]-7-methyl-7H-pyrrolo[2,3-d]pyrimidin-4-amine;
5-{4-fluoro-1-[(1-methyl-1H-pyrrol-2-yl)acetyl]-2,3-dihydro-1H-indol-5-yl}-7-methyl-7H-pyrrolo[2,3-d]pyrimidin-4-amine;
5-{1-[(2,5-difluorophenyl)acetyl]-4-fluoro-2,3-dihydro-1H-indol-5-yl}-7-methyl-7H-pyrrolo[2,3-d]pyrimidin-4-amine;
5-{1-[(2,5-difluorophenyl)acetyl]-2,3-dihydro-1H-indol-5-yl}furo[2,3-d]pyrimidin-4-amine;
5-(1-{[3-(trifluoromethyl)phenyl]acetyl}-2,3-dihydro-1H-indol-5-yl)furo[2,3-d]pyrimidin-4-amine;
5-{1-[(3-chloro-5-fluorophenyl)acetyl]-2,3-dihydro-1H-indol-5-yl}furo[2,3-d]pyrimidin-4-amine;
5-{1-[(3-methylphenyl)acetyl]-2,3-dihydro-1H-indol-5-yl}furo[2,3-d]pyrimidin-4-amine;
5-(1-{[3-fluoro-5-(trifluoromethyl)phenyl]acetyl}-2,3-dihydro-1H-indol-5-yl)furo[2,3-d]pyrimidin-4-amine;
5-{1-[(2,5-difluorophenyl)acetyl]-2,3-dihydro-1H-indol-5-yl}-7-[2-(4-piperidinyl)ethyl]-7H-pyrrolo[2,3-d]pyrimidin-4-amine;
7-methyl-5-{1-[(6-methyl-2-pyridinyl)acetyl]-2,3-dihydro-1H-indol-5-yl}-7H-pyrrolo[2,3-d]pyrimidin-4-amine;
5-(1-{[4-fluoro-3-(trifluoromethyl)phenyl]acetyl}-2,3-dihydro-1H-indol-5-yl)-7-methyl-7H-pyrrolo[2,3-d]pyrimidin-4-amine;
5-{1-[(2,5-difluorophenyl)acetyl]-2,3-dihydro-1H-indol-5-yl}-7-(3-oxetanyl)-7H-pyrrolo[2,3-d]pyrimidin-4-amine;
3-{1-[(2,5-difluorophenyl)acetyl]-2,3-dihydro-1H-indol-5-yl}-7-[2-(dimethylamino)ethyl]furo[3,2-c]pyridin-4-amine;
7-methyl-5-(1-{[6-(trifluoromethyl)-2-pyridinyl]acetyl}-2,3-dihydro-1H-indol-5-yl)-7H-pyrrolo[2,3-d]pyrimidin-4-amine;
7-(3-oxetanyl)-5-(1-{[3-(trifluoromethyl)phenyl]acetyl}-2,3-dihydro-1H-indol-5-yl)-7H-pyrrolo[2,3-d]pyrimidin-4-amine;
7-[2-(4-morpholinyl)ethyl]-5-(1-{[3-(trifluoromethyl)phenyl]acetyl}-2,3-dihydro-1H-indol-5-yl)-7H-pyrrolo[2,3-d]pyrimidin-4-amine;
7-(1-methylethyl)-5-(1-{[3-(trifluoromethyl)phenyl]acetyl}-2,3-dihydro-1H-indol-5-yl)-7H-pyrrolo[2,3-d]pyrimidin-4-amine;
7-(3-methylbutyl)-5-(1-{[3-(trifluoromethyl)phenyl]acetyl}-2,3-dihydro-1H-indol-5-yl)-7H-pyrrolo[2,3-d]pyrimidin-4-amine;
4-{1-[(3-methylphenyl)acetyl]-2,3-dihydro-1H-indol-5-yl}-1H-pyrazolo[3,4-c]pyridin-3-amine;
7-chloro-3-{1-[(2,5-difluorophenyl)acetyl]-2,3-dihydro-1H-indol-5-yl}furo[3,2-c]pyridin-4-amine;
7-(3-azetidinyl)-5-(1-{[3-(trifluoromethyl)phenyl]acetyl}-2,3-dihydro-1H-indol-5-yl)-7H-pyrrolo[2,3-d]pyrimidin-4-amine;
7-(1-methyl-3-azetidinyl)-5-(1-{[3-(trifluoromethyl)phenyl]acetyl}-2,3-dihydro-1H-indol-5-yl)-7H-pyrrolo[2,3-d]pyrimidin-4-amine;
7-[2-(dimethylamino)ethyl]-5-(1-{[3-(trifluoromethyl)phenyl]acetyl}-2,3-dihydro-1H-indol-5-yl)-7H-pyrrolo[2,3-d]pyrimidin-4-amine;
5-(4-fluoro-1-{[3-(trifluoromethyl)phenyl]acetyl}-2,3-dihydro-1H-indol-5-yl)-7-methyl-7H-pyrrolo[2,3-d]pyrimidin-4-amine;
5-{4-fluoro-1-[(6-methyl-2-pyridinyl)acetyl]-2,3-dihydro-1H-indol-5-yl}-7-methyl-7H-pyrrolo[2,3-d]pyrimidin-4-amine;
5-(4-fluoro-1-{[6-(trifluoromethyl)-2-pyridinyl]acetyl}-2,3-dihydro-1H-indol-5-yl)-7-methyl-7H-pyrrolo[2,3-d]pyrimidin-4-amine;
5-{1-[(3,5-dimethyl-1H-pyrazol-1-yl)acetyl]-4-fluoro-2,3-dihydro-1H-indol-5-yl}-7-methyl-7H-pyrrolo[2,3-d]pyrimidin-4-amine;
5-(4-fluoro-1-{[4-fluoro-3-(trifluoromethyl)phenyl]acetyl}-2,3-dihydro-1H-indol-5-yl)-7-methyl-7H-pyrrolo[2,3-d]pyrimidin-4-amine;
3-{1-[(2,5-difluorophenyl)acetyl]-4-fluoro-2,3-dihydro-1H-indol-5-yl}furo[3,2-c]pyridin-4-amine;
5-{4-fluoro-1-[(4-fluorophenyl)acetyl]-2,3-dihydro-1H-indol-5-yl}-7-methyl-7H-pyrrolo[2,3-d]pyrimidin-4-amine;
4-(1-{[3-(trifluoromethyl)phenyl]acetyl}-2,3-dihydro-1H-indol-5-yl)-1H-pyrazolo[3,4-c]pyridin-3-amine;
1-methyl-4-(1-{[3-(trifluoromethyl)phenyl]acetyl}-2,3-dihydro-1H-indol-5-yl)-1H-pyrazolo[3,4-c]pyridin-3-amine;
7-(3-azetidinyl)-5-{1-[(2,5-difluorophenyl)acetyl]-2,3-dihydro-1H-indol-5-yl}-7H-pyrrolo[2,3-d]pyrimidin-4-amine;
7-[2-(4-piperidinyl)ethyl]-5-(1-([3-(trifluoromethyl)phenyl)acetyl]-2,3-dihydro-1H-indol-5-yl)-7H-pyrrolo[2,3-d]pyrimidin-4-amine;
7-(2-aminoethyl)-3-{1-[(2,5-difluorophenyl)acetyl]-4-fluoro-2,3-dihydro-1H-indol-5-yl}furo[3,2-c]pyridin-4-amine;
3-{1-[(3,5-dimethyl-1H-pyrazol-1-yl)acetyl]-2,3-dihydro-1H-indol-5-yl}-1-methyl-1H-pyrazolo[3,4-d]pyrimidin-4-amine;
5-(1-{[3-(trifluoromethyl)phenyl]acetyl}-2,3-dihydro-1H-indol-5-yl)-1H-pyrrolo[2,3-d]pyrimidin-4-amine;
5-{4-chloro-1-[(6-methyl-2-pyridinyl)acetyl]-2,3-dihydro-1H-indol-5-yl}-7-methyl-7H-pyrrolo[2,3-d]pyrimidin-4-amine; and
5-(4-chloro-1-{[6-(trifluoromethyl)-2-pyridinyl]acetyl}-2,3-dihydro-1H-indol-5-yl)-7-methyl-7H-pyrrolo[2,3-d]pyrimidin-4-amine;
and salts thereof, including pharmaceutically acceptable salts thereof.

12. (canceled)

13. siRNA directed against the nucleic acid transcribed from the PERK gene.

14. The siRNA of claim 13 having the sequence 5′-CAAACUGUAUAACGGUUUATT-3′.

15. An antibody, or a functional variant thereof, or a fragment of the antibody capable of binding to PERK protein.

16. (canceled)

17. The method of claim 1, further comprising administration of a peroxisome proliferator-activated receptor (PPAR) agonist and/or an anti-diabetic agent.

18. (canceled)

19. (canceled)

20. The method of claim 1, further comprising administration of an agent used in the treatment of diabetes, obesity or insulin resistance syndrome.

21. (canceled)

22. (canceled)

23. (canceled)

24. (canceled)

25. (canceled)

26. (canceled)

27. A pharmaceutical composition comprising an inhibitor of PERK gene or a functional variant thereof, or an inhibitor of PERK protein or a functional variant thereof.

28. (canceled)

29. A method of identifying a compound that modulates expression of PERK gene in a cell, the method comprising:
a. exposing cells expressing the PERK gene with a test compound;
b. determining the expression level of the PERK gene in the cells which were exposed to the test compound under (a);
c. comparing the level of expression of the PERK gene determined under (b) with the expression of the PERK gene in control cells which were not exposed to the test compound; wherein a difference in the expression level between the cells under (b) compared to the control cells identifies the compound that modulates expression of the PERK gene in a cell.

30. A method of identifying a compound that modulates the amount or activity of PERK protein comprised in a cell, the method comprising:
a. exposing cells expressing PERK protein with a test compound;
b. determining the amount or activity of PERK protein in the cells which were exposed to the test compound under (a);
c. comparing the amount or activity of PERK protein determined under (b) with the activity of PERK protein in control cells not exposed to the test compound; wherein a difference in the amount or activity of PERK protein between the cells under (b) compared to the control cells identifies the compound that modulates the amount of PERK protein in the cells.

31. A method of identifying a compound that modulates the amount of PERK protein comprised in a cell, the method comprising:
a. exposing cells expressing PERK protein with a test compound;
b. determining the amount of PERK protein in the cells which were exposed to the test compound under (a);
c. comparing the amount of PERK protein determined under (b) with the amount of PERK in control cells not exposed to the test compound; wherein a difference in the amount of PERK protein between the cells under (b) compared to the control cells identifies the compound that modulates the amount of PERK protein in the cells.

32. A prognostic method for determining the receptiveness of a patient suffering from insulin resistance syndrome for a treatment with a PERK inhibitor, wherein the method comprises identifying and determining the PERK activity in the patient, wherein an increased PERK activity indicates that the person may be receptive for a treatment with a PERK inhibitor.

33. (canceled)

34. A prognostic method for determining the receptiveness of a patient suffering from insulin resistance syndrome for a treatment with a PERK inhibitor, wherein the method comprises measuring activity of a protein kinase AKT and/or PI3Kinase activity in a subject, wherein in comparison to a control a lowered AKT activity and/or lowered PI3Kinase activity indicates that the patient may be receptive for a treatment with a PERK inhibitor.

35. (canceled)

36. A prognostic method for determining the receptiveness of a patient suffering from insulin resistance syndrome for a treatment with a PERK inhibitor, wherein the method comprises measuring the relative levels of phosphorylation on the AKT site and on the PERK site(s), wherein a lower ratio of AKT site phosphorylation to PERK site phosphorylation indicates that the patient is receptive for a treatment with a PERK inhibitor.

37. (canceled)

38. A kit for use in treating or preventing insulin resistance syndrome in a patient, said kit comprises one of the following selected from the group consisting of a siRNA directed against the nucleic acid transcribed from the PERK gene, an antibody, or a functional variant thereof, or a fragment of the antibody capable of binding to PERK protein and a pharmaceutical composition comprising an inhibitor of PERK gene or a functional variant thereof, or an inhibitor of PERK protein or a functional variant thereof.

39. A kit for determining whether a patient suffering from insulin resistance syndrome is receptive for a treatment with a PERK inhibitor, wherein the kit comprises:
a. antibodies specific to one or more of the AKT phosphorylation site(s) on one or more of the human FOXO proteins; and
b. antibodies specific for one or more of the PERK phosphorylation site(s) on one or more of the human FOXO proteins or for one or more of the PERK phosphorylation site(s) on PERK protein or on elF2α.

40. (canceled)

41. (canceled)
